(19) United States
(12) Patent Application Publication
(10) Pub. No.: US 2016/0193231 A1
(43) Pub. Date: Jul. 7, 2016

(54) RETT SYNDROME AND TREATMENTS THEREFORE

(71) Applicants: RETT SYNDROME RESEARCH TRUST, Trumbull, CT (US); BAYLOR COLLEGE OF MEDICINE, Houston, TX (US)

(72) Inventors: David Scheer, Trumbull, CT (US); Monica J. Justice, Houston, TX (US)

(21) Appl. No.: 14/392,297

(22) PCT Filed: Jun. 26, 2014
(86) PCT No.: PCT/US14/44449

§ 371 Date: Dec. 23, 2015

Related U.S. Application Data
(60) Provisional application No. 61/839,759, filed on Jun. 26, 2013.

(51) Int. Cl. A61K 31/575; A61K 31/40; A61K 31/505; A61K 31/404; A61K 49/00; A61K 31/192; A61K 31/155; A61K 31/4439; A61K 31/427; A61K 31/366; A61K 31/426

(52) U.S. Cl. CPC A61K 31/575; A61K 31/366; A61K 31/40; A61K 31/505; A61K 31/404; A61K 31/426; A61K 31/192; A61K 31/155; A61K 31/4439; A61K 31/427; A61K 49/0004

(57) ABSTRACT

The present invention provides new strategies for the treatment of Rett Syndrome and other MECP2-associated disorders, including for the identification and/or characterization of useful therapeutic modalities and/or for the stratification of Rett Syndrome patients to identify those more or less likely to respond to a particular therapy. In some embodiments, the present invention defines certain components of metabolic pathways, and particularly of lipid and/or cholesterol metabolism (e.g., biosynthesis) pathways, most particularly of lipid and/or cholesterol metabolism (e.g., biosynthesis) pathways in the brain, as targets useful in the identification and/or characterization of potential Rett Syndrome treatment agents. Among other things, the present invention provides systems for identifying and/or characterizing such agents by contacting them with a system that comprises one or more such metabolic pathway components, and assessing their impact on presence, level, activity, and/or form of one or more indicators (e.g., components, products, and/or markers of the relevant pathway(s)).

### BACKGROUND

Rett Syndrome (RTT) is an X-linked disorder characterized by progressive development of motor and neurological dysfunction. Girls affected with RTT acquire speech and movement skills on a normal timeline after birth, but develop symptoms between 6 months and 2 years of age. Neurological manifestations of disease include: loss of speech and motor skills, stereotypic hand movements, difficulty walking, sporadic rapid respiration and apnea, and seizures. The prevalence of RTT is high (1 in 10,000 births), and it is one of the most common causes of intellectual and developmental disabilities (IDD) in females. Lifespan and disease severity vary greatly.

More than 95% of RTT patients carry a mutation in the MECP2 gene. Mechanistically, MECP2 binds to methylated DNA to regulate gene transcription through repression or activation. When MECP2 represses gene transcription, it associates with chromatin-remodeling complexes that contain Type I histone deacetylases (HDACs) (Bienvenu and Chelly Nat Rev Genet 7: 415-426 2006). Therefore, the elimination of MECP2 may result in the upregulation of genes that would normally be repressed. Notably, the severity of MECP2 mutation does not always correlate with disease severity, due at least in part to favorable patterns of X-chromosome inactivation in heterozygous females.

Mouse models that carry Mecp2 mutations and recapitulate most of the symptoms of RTT are available. Mecp2/Y male mutant mice are normal at birth and weaning, but develop symptoms that include hypo-activity, limb clasping, tremors, motor impairment and abnormal breathing as early as 4 weeks of age. Such symptoms progressively worsen, leading to their death at 6-16 weeks. Pronounced neuronal deficits are observed in Mecp2/Y null mice, including delayed transition into mature stages, altered expression of presynaptic proteins and reduced dendritic spine density.

Remarkably, re-expression of Mecp2 in mutant male and female mice after the onset of symptoms rescues neurological deficits and mice recover normal movements to live a long life (Guy et al Science 315: 1143-1147, 2007). These findings show that RTT is not caused by a permanent abnormal development of neurons during embryogenesis; instead, MECP2 is required for the maintenance of neurons after birth. Thus, RTT may be ameliorated or even reversed by genetic or pharmacological means after the onset of symptoms, providing tremendous hope for patients and families. Unfortunately, gene therapy using MECP2 is challenging, because brain function is exquisitely sensitive to levels of MECP2: increased MECP2 causes a progressive neurological disorder that leads to death as well (Bienvenu and Chelly Nat Rev Genet 7: 415-426 2006). There remains a critical need for the identification and development of new treatments for Rett Syndrome.

### SUMMARY

The present invention provides new strategies for the treatment of Rett Syndrome, including for the identification and/or characterization of useful therapeutic modalities and/or for the stratification of Rett Syndrome patients to identify those more or less likely to respond to a particular therapy.

In some embodiments, the present invention defines certain components of metabolic pathways, and particularly of lipid and/or cholesterol metabolism (e.g., biosynthesis) pathways, most particularly of lipid and/or cholesterol metabolism (e.g., biosynthesis) pathways in the brain, as targets useful in the identification and/or characterization of potential Rett Syndrome treatment agents. Among other things, the present invention provides systems for identifying and/or characterizing such agents by contacting them with a system that comprises one or more such metabolic pathway components, and assessing their impact on presence, level, activity, and/or form of one or more indicators (e.g., components, products, and/or markers of the relevant pathway(s)). In some embodiments, a provided system comprises a complete and/or active metabolic pathway (e.g., a lipid or cholesterol biosynthesis pathway). In some embodiments, a system includes or produces squalene monooxygenase. In some embodiments, a system includes or produces 24S-hydroxycholesterol (24S-OHC). In some embodiments, 24C-OHC may be utilized as an indicator, for example of metabolic pathway activity. In some embodiments, 24C-OHC may be assessed (e.g., by determining its presence, level, activity, and/or form) in a sample (e.g., a tissue sample such as a blood sample) from a subject.

In some embodiments, provided identification and/or characterization systems comprise one or more cells, tissues, and/or organisms. In some embodiments, such systems are or comprise mouse cells, tissues, and/or organisms. In some embodiments, such systems are or comprise one or more mouse cells, tissues, and/or organisms that show reduced expression and/or activity of MECP2 (e.g., as a result of genetic mutation and/or chemical alteration).

In some embodiments, the present invention provides methods and/or compositions for the treatment of Rett Syndrome. In some embodiments, provided methods and/or compositions include or utilize one or more agents that modulates MECP2 function or activity (i.e., MECP2 modulators). In some embodiments provided methods and/or compositions include or utilize one or more agents that modulate lipid and/or cholesterol metabolism (e.g., biosynthesis) pathways, and particularly lipid and/or cholesterol metabolism pathways in the brain.

In some embodiments, the present invention provides methods of treating a MECP2-associated disease, disorder, or condition including a step of administering at least one agent or modality that modulates lipid and/or cholesterol metabolism in the brain to a subject in need thereof. In some embodiments, the at least one agent or modality is selected from: a statin, an LXR modulator, a glucose metabolism modulator, a SREBP modulator, a PPARG modulator, and combinations thereof.

In some particular embodiments, the present invention provides methods of treating Rett Syndrome, which methods include a step of administering a statin to a subject suffering from or susceptible to Rett Syndrome. In some embodiments, the present invention provides methods of treating Rett Syndrome, which methods include a step of administering a glucose metabolism modulator to a subject suffering from or susceptible to Rett Syndrome.

In some embodiments, the present invention provides methods of treating a MECP2-associated disease, disorder, or condition (e.g., Rett Syndrome) by administering an agent or modality (e.g., a statin or glucose metabolism modulator) is administered at least once per day. In some embodiments, an agent or modality (e.g., a statin or glucose metabolism modulator) is administered at least once a week. In some embodiments, an agent or modality (e.g., a statin or glucose metabolism modulator) is administered at least twice a week. In some embodiments, an agent or modality (e.g., a statin or glucose metabolism modulator) is administered subcutaneously, intraperitoneally, intravenously, or orally.

In some embodiments, a statin for use in accordance with the present invention is or comprises at least one of lovastatin, simvastatin, atorvastatin, rosuvastatin, pravastatin, pitavastatin, and fluvastatin.

In some embodiments, an LXR modulator for use in accordance with the present invention is or comprises at least one of an oxysterol, a LXR agonist, an RXR agonist, and combinations thereof. In some embodiments, an LXR modulator is or comprises at least one of hypocholamide, T0901317, GW3965, SR9238, 22(R)-hydroxycholesterol, 24(S)-hydroxycholesterol, 27-hydroxycholesterol, cholestenoic acid, bexarotene, and combinations thereof.

In some embodiments, a glucose metabolism modulator for use in accordance with the present invention is or comprises at least one of a biguanide drug, 2,4-dinitrophenol-methyl ether (DNP-ME), 2,4-dinitrophenol-ethyl ether (DNP-EE), 2,4-dinitrophenol-vinyl ether (DNP-VE), and combinations thereof.

In some embodiments, a biguanide drug for use in accordance with the present invention is or comprises at least one of metformin, proguanil, chlorproguanil, and combinations thereof.

In some embodiments, an SREBP modulator for use in accordance with the present invention is or comprises at least one of fatostatin, N-(4-(2-(2-propylpyridin-4-yl)thiazol-4-yl)phenyl)methanesulfonamide (FGH10019), SREBP1, SREBP2, and combinations thereof.

In some embodiments, a PPARG modulator for use in accordance with the present invention is or comprises a thiazolidinedione. In some embodiments, a thiazolidinedione is or comprises at least one of rosiglitazone, pioglitazone, troglitazonc, netoglitazone, rivoglitazone, ciglitazone, and combinations thereof.

In some aspects, the present invention provides methods of identifying and/or characterizing useful therapeutic agents for the treatment of Rett Syndrome. In some embodiments, such methods may include a step of determining effect(s) of a candidate therapeutic agent on one or more aspects of lipid and/or cholesterol metabolism in the brain.

In some embodiments, aspects of lipid and/or cholesterol metabolism relevant to practice of the present invention may be or include cholesterol biosynthesis. In some embodiments, the one or more aspects of lipid and/or cholesterol metabolism is inhibition of 3-hydroxy-3-methyl-glutaryl-CoA reductase (HMGCR). In some embodiments, the one or more aspects of lipid and/or cholesterol metabolism is inhibition of squalene monooxygenase also known as squalene epoxidase (SQLE). In some embodiments, the one or more aspects of lipid and/or cholesterol metabolism is production of 24S-OHC.

In some embodiments, effect(s) of agents on one or more aspects of lipid and/or cholesterol metabolism is assessed via one or more of a: behavioral test, cognitive test, motor function test, test of one or more physiological parameters, and combinations thereof.

In some embodiments, a behavioral test useful in accordance with the present invention is selected from: acoustic startle response test, pre-pulse inhibition of startle response test, open field activity test, three chamber social interaction test, Home Cage Activity test, and/or combinations thereof.

In some embodiments, a motor function test useful in accordance with the present invention is selected from: breathing challenge, rotarod test, open field locomotor activity test, DigiGait system (Mouse Specifics) and combinations thereof.

In some embodiments, a test of one or more physiological parameters useful in accordance with the present invention is selected from: dual X-ray absorptiometry (DEXA) test, whole body plethysmography breathing test with methacholine challenge, glucose tolerance test, insulin tolerance test, serum cholesterol test, calorimetry test, and combinations thereof.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### Definitions

In order for the present disclosure to be more readily understood, certain terms are defined below. Additional definitions for, or clarifications of, the following terms and other terms may be set forth throughout the specification.

In this application, the use of “or” means “and/or” unless stated otherwise. As used in this application, the term “comprise” and variations of the term, such as “comprising” and “comprises,” are used in situations where listed items, elements, or steps are included and others may also be included. As used in this application, the terms “about” and “approximately” are used as equivalents. Any numerals used in this application, whether or not preceded by “about” or “approximately” are meant unless otherwise indicated to cover any normal fluctuations (e.g., standard errors or deviations), as would be appreciated by one of ordinary skill in the relevant art. In certain embodiments, the terms “approximately” or “about” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administration: As used herein, the term “administration” refers to the administration of a composition/agent to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

Animal: As used herein, the term “animal” refers to any member of the animal kingdom. In some embodiments, “animal” refers to humans, at any stage of development. In some embodiments, “animal” refers to non-human animals, at any stage of development. In sonic embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, IgG4], IgM) are defined based on the class of the utilized heavy chain sequences. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is monoclonal; in some embodiments, an antibody is polyclonal. In some embodiments, an antibody has constant region sequences that are characteristic of mouse, rabbit, primate, or human antibodies. In some embodiments, an antibody sequence elements are humanized, primatized, chimeric, etc, as is known in the art. Moreover, the term “antibody” as used herein, will be understood to encompass (unless otherwise stated or clear from context) can refer in appropriate embodiments to any of the art-known or developed constructs or formats for capturing antibody structural and functional features in alternative presentation. For example, in some embodiments, the term can refer to bi- or other multi-specific (e.g., zybodies, etc) antibodies, Small Modular ImmunoPharmaceuticals (“SMIPs™”), single chain antibodies, cameloid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc]).

Antibody fragment: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and CDR-containing moieties included in multi-specific antibodies formed from antibody fragments. Those skilled in the art will appreciate that the term “antibody fragment” does not imply and is not restricted to any particular mode of generation. An antibody fragment may be produced through use of any appropriate methodology, including but not limited to cleavage of an intact antibody, chemical synthesis, recombinant production, etc.

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another arc covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biocompatible: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

Biodegradable: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Biologically active: As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

Combination therapy: As used herein, the term “combination therapy” refers to those situations in which a subject is simultaneously exposed to two or more therapeutic agents. In some embodiments, such agents are administered simultaneously; in some embodiments, such agents are administered sequentially; in some embodiments, such agents are administered in overlapping regimens.

Comparable: The term “comparable”, as used herein, refers to two or more agents, entities, situations, sets of conditions, etc that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc to be considered comparable.

Corresponding to: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190^{th} residue in the first polymer but rather corresponds to the residue found at the 190^{th} position in the reference polymer; those of ordinary skill in the art readily appreciate how to identify “corresponding” amino acids, including through use of one or more commercially-available algorithms specifically designed for polymer sequence comparisons.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Dosage form: As used herein, the term “dosage form” refers to a physically discrete unit of a therapeutic agent for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated Runt one another by a time period of the same length; in some embodiments, a dosing regimen comprises a plurality of doses and at least two different time periods separating individual doses. In some embodiments, a dosing regimen is correlated with a desired or beneficial outcome when administered across a relevant population (i.e., is a therapeutic dosing regimen).

Encapsulated: The term “encapsulated” is used herein to refer to substances that are completely surrounded by another material.

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the whole.

Functional: As used herein, the term “functional” is used to refer to a form or fragment of an entity that exhibits a particular property and/or activity.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methioninc | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. In some embodiments, isolation involves or requires disruption of covalent bonds (e.g., to isolate a polypeptide domain from a longer polypeptide and/or to isolate a nucleotide sequence element from a longer oligonucleotide or nucleic acid).

MECP2 Modulator: The term “MECP2 Modulator”, as used herein, refers to an agent whose presence, level, state and/or form correlates with an alteration in MECP2 level and/or activity. That is, observed MECP2 level and/or activity is detectably different in the presence of the agent (or when the agent is at a particular level, or in a particular state or form) as compared to its absence and/or as compared to a comparable reference.

Modulator: In general, the term “modulator” is used to refer to an entity whose presence, level, state, and/or form in a system in which an activity of interest is observed correlates with a change in level and/or nature of that activity as compared with that observed under otherwise comparable conditions when the modulator (or its relevant level, state and/or form) is absent. In some embodiments, a modulator is an activator, in that activity is increased in its presence as compared with its absence under otherwise comparable conditions when the modulator is absent. In some embodiments, a modulator is an inhibitor, in that activity is reduced in its presence as compared with its absence under otherwise comparable conditions. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level. In some embodiments, activity of a modulator is assessed relative to a reference; in some embodiments, such reference may be a historical reference and/or may be embodied in a tangible or electronic medium.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Patient: As used herein, the term “patient” or “subject” refers to a human or any non-human animal (e.g., mouse, rat, rabbit, dog, cat, cattle, swine, sheep, horse or primate) to whom therapy is administered. In many embodiments, a patient is a human being. In some embodiments, a patient is a human presenting to a medical provider for diagnosis or treatment of a disease, disorder or condition. In some embodiments, a patient displays one or more symptoms or characteristics of a disease, disorder or condition. In some embodiments, a patient does not display any symptom or characteristic of a disease, disorder, or condition. In some embodiments, a patient is someone with one or more features characteristic of susceptibility to or risk of a disease, disorder, or condition.

Pharmaceutically acceptable: The term “pharmaceutically acceptable” as used herein, refers to agents that, within the scope of sound medical judgment, are suitable for use in contact with tissues of human beings and/or animals without excessive toxicity, irritation, allergic response, or other problem or complication, commensurate with a reasonable benefit/risk ratio.

Polypeptide: The term “polypeptide”, as used herein, generally has its art-recognized meaning of a polymer of at least three amino acids, linked to one another by peptide bonds. In some embodiments, the term is used to refer to specific functional classes of polypeptides, such as, for example, autoantigen polypeptides, nicotinic acetylcholine receptor polypeptides, alloantigen polypeptides, etc. For each such class, the present specification provides several examples of amino acid sequences of known exemplary polypeptides within the class; in some embodiments, such known polypeptides are reference polypeptides for the class. In such embodiments, the term “polypeptide” refers to any member of the class that shows significant sequence homology or identity with a relevant reference polypeptide. In many embodiments, such member also shares significant activity with the reference polypeptide. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region, often including a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide as described herein may comprise or consist of a fragment of a parent polypeptide. In some embodiments, a useful polypeptide as described herein may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: The term “reference” is often used herein to describe a standard or control agent or value against which an agent or value of interest is compared. In some embodiments, a reference agent is tested and/or a reference value is determined substantially simultaneously with the testing or determination of the agent or value of interest. In some embodiments, a reference agent or value is a historical reference, optionally embodied in a tangible medium. Typically, as would be understood by those skilled in the art, a reference agent or value is determined or characterized under conditions comparable to those utilized to determine or characterize the agent or value of interest.

Sample: The term “sample” refers to a volume or mass obtained, provided, and/or subjected to analysis. In some embodiments, a sample is or comprises a tissue sample, cell sample, a fluid sample, and the like. In some embodiments, a sample is taken from a subject (e.g., a human or animal subject) In some embodiments, a tissue sample is or comprises brain, hair (including roots), buccal swabs, blood, saliva, semen, muscle, or from any internal organs, or cancer, precancerous, or tumor cells associated with any one of these. A fluid may be, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g., a subject); in some embodiments, a “sample” is the result of processing of a primary sample, for example to remove certain potentially contaminating components and/or to isolate or purify certain components of interest.

Small molecule: As used herein, the term “small molecule” means a low molecular weight organic compound that may serve as an enzyme substrate or regulator of biological processes. In general, a “small molecule” is a molecule that is less than about 5 kilodaltons (kD) in size. In some embodiments, provided nanoparticles further include one or more small molecules. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, one or more small molecules are encapsulated within the nanoparticle. In some embodiments, small molecules are non-polymeric. In some embodiments, in accordance with the present invention, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition has been diagnosed with and/or exhibits or has exhibited one or more symptoms or characteristics of the disease, disorder, or condition.

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from allergy, etc.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Therapeutic agent: As used herein, the phrase “therapeutic agent” refers to any agent that has a therapeutic effect and/or elicits a desired biological and/or pharmacological effect, when administered to a subject. In some embodiments, an agent is considered to be a therapeutic agent if its administration to a relevant population is statistically correlated with a desired or beneficial therapeutic outcome in the population, whether or not a particular subject to whom the agent is administered experiences the desired or beneficial therapeutic outcome.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweart, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Therapeutic regimen: A “therapeutic regimen”, as that term is used herein, refers to a dosing regimen whose administration across a relevant population is correlated with a desired or beneficial therapeutic outcome.

Treatment: As used herein, the term “treatment” (also “treat” or “treating”) refers to any administration of a substance that partially or completely alleviates, ameliorates, relives, inhibits, delays onset of, reduces severity of, and/or reduces frequency, incidence or severity of one or more symptoms, features, and/or causes of a particular disease, disorder, and/or condition. Such treatment may be of a subject who does not exhibit signs of the relevant disease, disorder and/or condition (e.g., may be prophylactic) and/or of a subject who exhibits only early signs of the disease, disorder, and/or condition. Alternatively or additionally, such treatment may be of a subject who exhibits one or more established signs of the relevant disease, disorder and/or condition (e.g., may be therapeutic). In some embodiments, treatment may be of a subject who has been diagnosed as suffering from the relevant disease, disorder, and/or condition in some embodiments, treatment may be of a subject known to have one or more susceptibility factors that are statistically correlated with increased risk of development of the relevant disease, disorder, and/or condition.

### BRIEF DESCRIPTION OF THE DRAWING

The Drawing, which is comprised of at least the following Figures, is for illustration purposes only, not for limitation.

FIG. 1 shows that a stop codon mutation in Sqle confers motor and longevity rescue. a) Survival of Mecp2^{tm1.1Bird}/Y mice is significantly increased by the presence of the Sqle^{Sum3Jus}/+ mutation; p=0.002. Mecp2^{tm1.1Bird}/Y Sqle^{Sum3}/+ animals at backcross generation N₇ to 129S6/SvEvTac show b) significantly improved rotarod performance at P56 (p=0.0001), c) improved open field activity at P70. Furthermore, the Sqle^{SumJus} mutation does not d) increase startle amplitude nor e) decrease time to startle in 129.Mecp2^{tm1.1Bird}/Y mice that undergo a pre-pulse inhibition assay at P70. nostim=No stimulus presented; as50=50 dB stimulus presented; pp8=8 dB pre-pulse presented; pp850=8 dB pre-pulse followed by 50 dB stimulus. All error bars represent s.e.m.

FIG. 2 shows that cholesterol metabolism is disrupted in Mecp2 null male mice. a) A simplified schematic of the enzymes and products in cholesterol biosynthesis via desmosterol is shown. b) Expression of Hmgcr, Sqle and Cyp46a1 in Mecp2^{tm1.1Bird}/Y and Mecp2^{tm1.1Jae}/Y are similar in brain. c) Lanosterol (Lan), desmosterol (Des) and total cholesterol (TC) concentrations are displayed per gram of brain tissue at P56 (N=8 per group) and P70 (N=4 per group). d) Cholesterol synthesis is decreased in Mecp2^{tm1.1Jae}/Y brain at P56 (wild type N=4; null N=5). e) Expression of Hmgcr and Sqle in Mecp2^{tm1.1Bird}/Y and Mecp2^{tm1.1Jae}/Y differ in liver. f) Triacylglyceride (TAG) and TC concentrations are displayed per gram of liver tissue at P56 (N=6 per group). g) Cholesterol synthesis is slightly increased in Mecp2^{tm1.1Jae}/Y liver per gram of tissue at P56 (wild type N=4; null N=5). Serum h) total cholesterol, i) LDL-cholesterol and j) triglyceride levels are significantly higher in Mecp2^{tm1.1Bird}/Y mice by D P56 (N=8-11 per group), but unchanged in Mecp2^{tm1.1Jae}/Y mice (N=6 per group). For gene expression data (b,e) Bird: N=6 per genotype at P28, and 12 per genotype at P56; Jae: N=4 per genotype at P28, and 6 per genotype at P56. Tissue data (b-g) represent percentage change from wild type levels. *p≦0.05; All error bars represent s.e.m.

FIG. 3 shows that Mecp2 mutant mice develop metabolic syndrome. As shown, both male and female Mecp2/Y and Mecp2/+ mice show an inability to clear glucose properly in an Intraperitoneal Glucose Tolerance Test (IPGTT), and show insulin resistance after a bolus injection of insulin (ITT). This metabolic dysregulation worsens as symptoms progress. Further, males burn fat rather than glucose during periods of activity. These are all signs of metabolic syndrome. Thus, the data presented in this FIG. 3 establish that Mecp2 null mice develop metabolic syndrome.

FIG. 4 shows that statin treatment improves health in 129.Mecp2^{tm1.1Bird}/Y males. Total animals assessed were 37 Mecp2^{tm1.1Bird}/Y fluvastatin-treated, 12 Mecp2^{tm1.1Bird}/Y lovastatin-treated, 31 Mecp2^{tm1.1Bird}/Y vehicle-treated, 29 wild type +/Y fluvastatin-treated, 8 +/Y lovastatin-treated, and 29 wild type +/Y vehicle-treated mice for the following tests. a) Fluvastatin treatment of 129.Mecp2^{tm1.1Bird}/Y confers increased longevity: median 122 days compared to 87 days with 57% survival beyond 120 days (p<0.0001). Three animals were sacrificed due to dermatitis (boxes) while active and otherwise healthy. b) Rotarod performance improves in P56 treated null males (fluvastatin p=0.015; lovastatin p=0.009), c) Open field activity is increased in P70 treated null males as assessed by beam breaks (fluvastatin: p=0.026, lovastatin: p=0.011). Furthermore, fluvastatin treatment does not d) increase startle amplitude nor e) decrease time to startle in 129.Mecp2^{tm1.1Bird}/Y mice that undergo a pre-pulse inhibition assay at P70. Statin treatment lowers plasma cholesterol by P70 (fluvastatin: p=0.001, lovastatin: p=0.001). g) Statin treatment ameliorates elevated lipid concentration in 129.Mecp2^{tm1.1Bird}/Y livers at P70 (fluvastatin p=0.02; lovastatin: p=0.386). The concentration of h) lanosterol slightly increases and i) desmosterol significantly increases in the brains of fluvastatin-treated 129.Mecp2^{tm1.1Bird}/Y mice at P70 (N=4 per group; p=0.04). j) shows histology of fatty liver before and after statin treatment.

FIG. 5: shows that Fluvastatin treatment improves health in 129.Mecp2^{tm1.1Bird}/+ females a) No fluvastatin-treated 129.Mecp2^{tm1.1Bird}/+ females died prior to eight months, but three vehicle-treated females died. b) Rotarod performance improves in five-month-old fluvastatin-treated 129.Mecp2^{tm1.1Bird}/+ females (p=0.001). c) Open field activity assessed at four months shows no significant differences in fluvastatin- or vehicle-treated groups. d) Fluvastatin treatment does not significantly change serum cholesterol levels at eight months. e) Fluvastatin treatment ameliorates elevated lipid concentration in 129.Mecp2^{tm1.1Bird}/+ livers assessed at eight months (p=0.045), f) shows histology Oil Red O of livers before and after statin treatment.

FIG. 6: shows an exemplary timeline for standard drug treatment protocol for Mecp2 male mice developed in the Justice laboratory. As shown, Females would receive a 1× weekly dose, and tests would be offset based on age. However, the tests would be the same. The timeline for females would start at 6 weeks, and end at 8 months, with rotarod being performed at 8 weeks, and the open field activity (OFA), Prepulse inhibition of acoustic startle response (PPI), plethysmography (Pleth), DEXA for body composition being carried out at 5 months, followed by a necropsy with all clinical lipid panels, and tissue lipids being assessed at necropsy at 6 months.

FIG. 7: shows exemplary graphs of treatment of mice using four different statin drugs of different lipophilicities. Rotarod and Open Field Activity (OFA) are measures of motor performance (panels B and C, respectively). Dual X-ray absorptiometry (DEXA) is a test for body fat and bone composition (panel E). Mice were administered a dose of 3 mg/kg fluvastatin, 2 mg/kg body weight of Atorvastatin, 1.5 mg/kg Lovastatin, or 6 mg/kg Simvastatin 2× per week and subjected to the behavior testing protocol shown in FIG. 6. The number of mice tested for each group is shown underneath the relevant bar. The dashed black line shows the wild type average, since none of the statin treatments changed wild type performance on any task. Data were analyzed using ANOVA followed by Dunnett PostHoc by comparing treatment to relevant control group. *p≦0.05, #p≦0.10

FIG. 8: shows exemplary graphs indicating that Mecp2 null mice cannot utilize glucose in peripheral organs. Hyperinsulinemic-Euglycemic clamps were performed at eight weeks of age on Mecp2 null and wild type littermates. Mecp2 null males require a significantly slower glucose infusion rate to maintain euglycemia (shown in panel A), suggesting an inability to metabolize glucose. Measurement of ¹⁴C glucose in white adipose tissue (WAT) and soleus muscle confirms decreased glucose uptake in the major glucose-consuming peripheral organs (shown in panels B and C, respectively).

FIG. 9: shows exemplary graphs after treatment of Mecp2/Y mice with an LXR agonist and metformin. The number of mice tested for each group is shown underneath the relevant bar. The dashed black line shows the wild type average, since none of the statin treatments changed wild type performance on any task. Mice were treated with a 30 mg/kg dose of metformin and a 3 mg/kg dose of the LXR agonist T0901917, 2× per week and analyzed per the protocol in FIG. 6. Data were analyzed using ANOVA followed by Dunnett PostHoc by comparing treatment to relevant control group. *p≦0.05, #p≦0.10

FIG. 10: shows exemplary graphs after treatment of Mecp2/Y mice with the glucose metabolism modulator, 2,4-dinitrophenol-methyl ether (DNPME). Mice were treated with a 5 mg/kg dose 2× weekly and analyzed per the protocol in FIG. 6. Wild type N=4, Mecp2/Y N=6, and 1 vehicle control null mouse died prior to P70. Data were analyzed using ANOVA followed by Bonferroni PostHoc by comparing treatment to relevant control group. *p≦0.05, #p≦0.10

### DETAILED DESCRIPTION OF CERTAIN EMBODIMENTS

MECP2-Associated Diseases, Disorders, and Conditions (e.g., Rett Syndrome)

The present invention relates to treatment of diseases, disorders, and conditions associated with disruption of MECP2 activity. MECP2 mutations are known to be associated with various diseases, disorders, and conditions. In various embodiments, the teachings of the present disclosure will be understood by those skilled in the art to be applicable to any disease, disorder or condition whose symptoms are associated and/or correlated with one or more MECP2 alterations (e.g., in level, activity, or form of MECP2 protein and/or with one or more particular mutations of the MECP2 gene).

Those skilled in the art will appreciate that the teachings of the present disclosure are particularly applicable to Rett Syndrome (RTT), noting that 95% of Rett Syndrome cases are associated with one or more MECP2 mutations. Rett Syndrome is an X-linked disorder which affects approximately one in ten-thousand girls. Patients go through four stages: Stage I) Following a period of apparently normal development from birth, the child begins to display social and communication deficits, similar to those seen in other autism spectrum disorders, between six and eighteen months of age. The child shows delays in their developmental milestones, particularly for motor ability, such as sitting and crawling. Stage II) Beginning between one and four years of age, the child goes through a period of regression in which they lose speech and motor abilities, developing stereotypical midline hand movements and gait impairments. Breathing irregularities, including apnea and hyperventilation also develop during this stage. Autistic symptoms are still prevalent at this stage. Stage III) Between age two and ten, the period of regression ends and symptoms plateau. Social and communication skills may show small improvements during this plateau period, which may last for most of the patients' lives. Stage IV) Motor ability and muscle deterioration continues. Many girls develop severe scoliosis and lose the ability to walk. Classic Rett Syndrome is monogenic, caused by mutations in MECP2.

Hemizygous human males with truncating or loss of function MECP2 mutations have more severe phenotypes than females with RTT, and usually die by 2 years of age. Hypomorphic mutations or duplications involving MECP2 are also associated with a variety of intellectual disability ID, autism, and other psychiatric features.

Furthermore, given that MECP2 is involved in cholesterol metabolism, in some embodiments, the present disclosure teaches that other components of one or more metabolic pathways associated with lipid and/or cholesterol biosynthesis may be considered to be MECP2-associated diseases, disorders, or conditions treatable with metabolic modulators as described herein. Cholesterol metabolism has been implicated in neurological diseases such as Alzheimer's, Parkinson's and Huntington's Diseases, as well as in Amyotrophic Lateral Sclerosis and Fragile X Syndrome. Also, in some embodiments, certain forms of autism, including in particular syndrome-associated autism associated with one or both of Rett Syndrome, may be considered MECP2-associated diseases, disorders or conditions as described herein. Autism, in its broadest sense is a genetically diverse group of disorders with complex etiologies, unlikely to be responsive to a single therapy.

In certain embodiments, the present invention describes treatment of any or all of these, and/or identification, characterization, and/or use of therapies and/or biomarkers for them.

In certain embodiments, the present invention provides methods of treating a MECP2-associated disease, disorder, or condition, which methods include a step of administering at least one agent or modality that modulates lipid and/or cholesterol metabolism in the brain to a subject in need thereof. In some embodiments, the at least one agent or modality is selected from: a statin, an LXR modulator, a glucose metabolism modulator, a SREBP modulator, a PPARG modulator, and combinations thereof.

### Metabolic Pathways

As described herein, the present invention encompasses the recognition that modulators of certain metabolic pathways (e.g., lipid and/or cholesterol biosynthesis pathways) may be useful in the treatment of Rett Syndrome and/or that systems comprising one or more components of such pathways may be useful in the identification and/or characterization of such modulators. The present invention also provides the insight that, in some instances, it may be useful to distinguish individual Rett Syndrome patients from one another on the basis of activity or character of one or more features of a metabolic pathway as described herein. Teachings of the present invention are particularly relevant to metabolic pathways involved in cholesterol and/or lipid biosynthesis in the brain and/or liver and/or other systemic metabolic components.

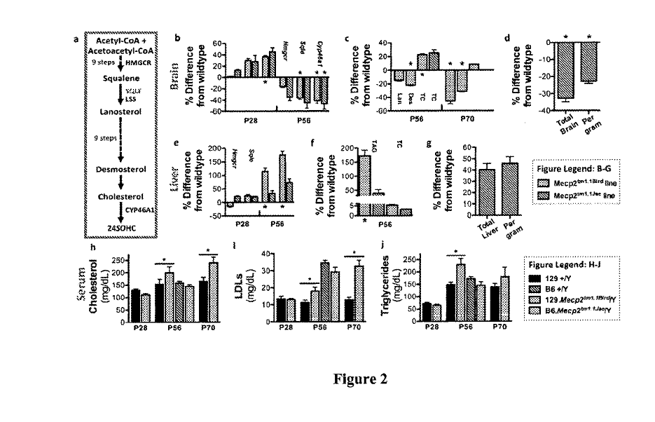

Cholesterol is a major component of the brain where it is synthesized through semi-independent pathways, which are identical through the conversion of squalene to lanosterol by squalene epoxidase (SQLE) and lanosterol synthase (LSS), because it cannot be supplied by dietary absorption or liver synthesis (Dietchy, Turley and Spady J Lipid Res 34:1637-1659, 1993) (FIG. 2a). Commonly, attention is placed on high circulating cholesterol in the blood, because it is associated with increased incidence to cardiovascular disease. However, cholesterol has many functions in neural tissues including membrane trafficking, signal transduction, myelin formation, dendrite remodeling, neuropeptide formation and synaptogenesis (Pfrieger and Ungerer, Prog Lipid Res 50: 357-371, 2011). Dysregulation of brain cholesterol metabolism can lead to accumulation of cholesterol protein intermediates that disrupt normal development when present in excess, and influence diseases of aging including Huntington and Alzheimer Disease (Waterham, FEBS Lett 580: 5442-5449, 2006).

Because of the role of cholesterol in cardiovascular disease, HMG CoA reductase inhibitors (statins) are medically prescribed for higher than normal cholesterol levels, or for elevated cholesterol levels that result in adverse effects. A normal level of cholesterol is a level that generally does not warrant therapeutic use of HMG CoA reductase inhibitors. The precise “normal” level may depend to some degree, as is understood in the art, on the subject and variations in cholesterol levels observed with respect to age, sex, diet and the population type. Generally, cholesterol levels are measured when a subject is not suffering from an acute illness, not under stress, and for a woman, when not pregnant.

In many embodiments, the level of cholesterol as used herein refers to the total serum cholesterol level, which includes the combined cholesterol found in sera in the form of high density lipoprotein (HDL), intermediate density lipoprotein (IDL), low density lipoprotein (LDL) and very low density lipoprotein (VLDL). Cholesterol levels are commonly measured in association with the storage form of lipids, triglycerides or triacylglyerol. A cholesterol level may be based on the amount of total cholesterol in the combined lipoprotein fraction. Cholesterol and triglycerides found in sera fractionate into various components: HDL, IDL, LDL, and VLDL. The LDL fraction derives from VLDL, and elevated levels of total serum cholesterol and cholesterol in the LDL (c-LDL) fraction are correlated with increased risk of atherosclerosis.

An exemplary normal serum cholesterol level for an adult human is a range that is below 200 mg/dL to about 140 mg/dL, is that considered healthy for the subject, depending on various factors, such as the age, diet and sex of the subject. A level considered healthy for a child or adolescent is between about 120 mg/dL and about 170 mg/dL. The population of subjects treatable using the methods herein include children or adolescents. The normal level of c-LDL for a human is less than about 150 mg/dL, less than about 130 mg/dL, or less than about 110 mg/dL with the lower limit being a level of c-LDL that is considered a healthy level. A level considered healthy for a child or adolescent is below 110 mg/dL.

Little is known about brain cholesterol metabolism, unlike peripheral metabolism. The present disclosure points to the importance of brain cholesterol homeostasis in Rett Syndrome for the first time. Because cholesterol is important for brain function, yet cannot cross the blood brain barrier (BBB), the brain manufactures its own cholesterol. However, it must recycle or turnover old cholesterol into the circulation and manufacture new, or the cholesterol can become oxidized and lead to inflammation. Cholesterol turnover is known to be required at the neuronal synapse; most cycling cholesterol in the adult brain not present in myelin is produced by astrocytes, packaged in HDL-like particles to be delivered through the intracellular space to LDL-like receptors on neurons (Pfrieger and Ungerer, Prog Lipid Res 50: 357-371, 2011). When neurons accumulate too much cholesterol or its intermediates, the cytochrome P450 oxidase Cyp46a1 hydroxylates cholesterol to produce 24SOHC, allowing for turnover by one-way diffusion into the circulation across the BBB (Lund, Guileyardo and Russell, Proc Natl Acad Sci USA 96: 7238-7243; Lund et al. J Biol Chem 278: 22980-22988, 2003).

### Metabolic Pathway Modulators

As described herein, the present invention encompasses the recognition that certain metabolic pathway modulators are useful in the treatment of Rett Syndrome and/or other MECP2-associated diseases, disorders, or conditions. In particular, the present invention establishes that certain modulators of lipid and/or cholesterol metabolism (e.g., biosynthesis) pathways, and particularly of lipid and/or cholesterol pathways in the brain are useful in the treatment of MECP2-associated diseases, disorders, or conditions. In some embodiments, the present invention teaches use of MECP2 modulators in the treatment of Rett Syndrome.

In general, metabolic pathway modulators useful as described herein may be or comprise any chemical class of agent including, for example, nucleic acid, polypeptide, lipid, carbohydrate, and/or small molecule agent, or combination thereof. Those skilled in the art, will appreciate, for example, that many protein targets can be inhibited by antibody agents and/or by targeted nucleic acid agents (e.g., antisense and/or siRNA agents). In some embodiments, a metabolic pathway inhibitor can cross the blood brain barrier (BBB).

### Statins

As described herein, statin drugs are useful in the treatment of MECP2-associated diseases, disorders, and conditions.

For example, the present Examples demonstrate that statin drugs recapitulate the amelioration of symptoms exhibited by the Sqle suppressor mutation in Mecp2 null mice. Under the particular conditions tested, the statins did not ameliorate all symptoms, including the acoustic startle response or prepulse inhibition of acoustic startle response.

Applicant notes that a publication by Silva (U.S. Patent Application publication number 2007/0299096, published Dec. 27, 2007 from U.S. patent application Ser. No. 11/569426, filed May 23, 2005 and claiming priority to U.S. Provisional Patent Applications with Ser. Nos. 60/574442 and 60/661764, filed May 24, 2004 and Mar. 15, 2005, respectively) entitled “Treating Learning Deficits with Inhibitors of HMG-CoA Reductase” (the “Silva Publication”) includes statements suggesting that inhibitors of the enzyme HMG-CoA Reductase, which catalyzes the conversion of HMG CoA to mevalonate, the isoprenoid intermediate used for cholesterol biosynthesis, might be generally useful for the treatment of cognitive disorders. At least some statins are believed to be HMG-CoA reductase inhibitors, and the Silva Publication specifically recommends use of statins for such treatment. However, the Silva Publication itself, consistent with the understanding in the art, also notes that cognition is a complicated neurological process, and that a diverse array of molecular mechanisms is implicated in cognitive function.

The Silva Publication defines a number of different biological pathways that might be involved in learning deficits associated with different diseases, disorders, or conditions. In particular, the Silva Publication highlights neurofibromin signaling pathways, as are involved in neurofibromatosis-1. The Silva Publication provides data showing modest beneficial impact of lovastatin on p21Ras/MAPK activity, long term potentiation, spatial learning deficits, and the attention and sensory gating deficit in a mouse model for neurofibromatosis-1 (“NF-1”).

The Silva Publication does not specifically recommend use of HMG-CoA reductase inhibitors in general, let alone statins in particular, in the treatment of Rett Syndrome. Indeed, Rett Syndrome itself is not among the cognitive disorders that the Silva Publication lists as properly treatable with HMG-CoA Reductase inhibitors. The Silva Publication does note, however, in its Background and its introduction, that some Rett Syndrome patients display autistic symptoms and/or share one or more genetic features with autistic individuals. The Silva Publication does list autism as a treatable disorder, although the biological pathways that it notes as relevant to development of autism are distinct from those associated with NF-1.

Unfortunately, clinical trials on NF-1 patients, motivated by the Silva Reference and designed to ameliorate learning disabilities and attention deficits, proved unsuccessful; simvastatin was not observed to show significant effect (see, for example, Krab et al. JAMA 300:287, 2008). Similarly, a clinical trial for Alzheimer's Disease using simvastatin failed (Sano et al. Neurology 77:556, 2011). The failure of these trials demonstrates that the NF-1 mouse and/or the associated cognitive tests carried out on the mice were not an appropriate model for human cognitive function. Such failure also suggests that the mechanism proposed in the Silva Reference—via modulation of Ras activity might not operate as suggested. Those skilled in the art would be familiar with these failures and would well understand their implications for the teaching of the Silva Reference.

The present disclosure, by contrast, demonstrates that statins can achieve improvements in motor deficits that may underlie neurological deficits, and furthermore establishes that statins' action lies in the ability to modulate lipid deposition. The present disclosure, so far as we are aware, represents the first teaching that lipid- or cholesterol-biosynthesis pathway modulators, and specifically that MECP2 modulators (e.g., statins) are usefiil in the treatment of Rett Syndrome. Certainly, the present disclosure is the first suggestion of such use in those Rett Syndrome patients who do not carry autism-associated genetic mutations (i.e., in loci other than MECP2).

Accordingly, in some embodiments, the present invention provides methods of treating a MECP-2 associated disease, disorder, or condition (e.g., Rett Syndrome), which methods include a step of administering a statin to a relevant subject suffering from or susceptible to the MECP-2 associated disease, disorder or condition. According to various embodiments, any statin may be used including, for example, atorvastatin, cerivastatin, fluvastatin, lovastatin, mevastatin, pitavastatin, pravastatin, rosuvastatin, simvastatin. In some embodiments, the statin is selected from: lovastatin, simvastatin, atorvastatin, fluvastatin, and combinations thereof.

### Liver Receptor X (LXR) Modulators

While statins treat high cholesterol by slowing the body's production of it, it is also possible to lower cholesterol levels by inducing the reuptake of blood cholesterol by the liver, where it can be converted into bile acids for excretion. This reuptake is mediated by LXR. LXR also has a brain specific role, although the exact mechanism is unknown. Drugs that directly modulate LXR activity include hypocholamide, T0901317, GW3965, SR9238 and bexarotene. These LXR agonists have been effective at treating mouse models of atherosclerosis and diabetes, and some compounds, particularly bexarotene, have been shown to cross the blood-brain barrier. Other drugs, such as Psck9 inhibitors, a number of which are currently under development indirectly affect LXR levels. Any of these are good candidates for an alternate or supplemental approach to statins for regulating cholesterol in models of Rett Syndrome and/or other MECP2-associated diseases, disorders, and conditions as described herein.

In some embodiments, an LXR modulator may be or comprise any oxysterol or RXR agonist. Non-limiting examples beyond those described above include, but are not limited to hypocholamide, 22(R)-hydroxycholesterol, 27-hyroxycholesterol, 24(S)-hydroxycholesterol (brain specific), 24(S), 25-epoxycholesterol (liver-specific), cholestenoic acid, and combinations thereof. The LXR agonists 5,6-24(S),25-diepoxycholesterol and 6alpha-hydroxy bile acids are selective for LXR alpha.

### Glucose Metabolism Modulators

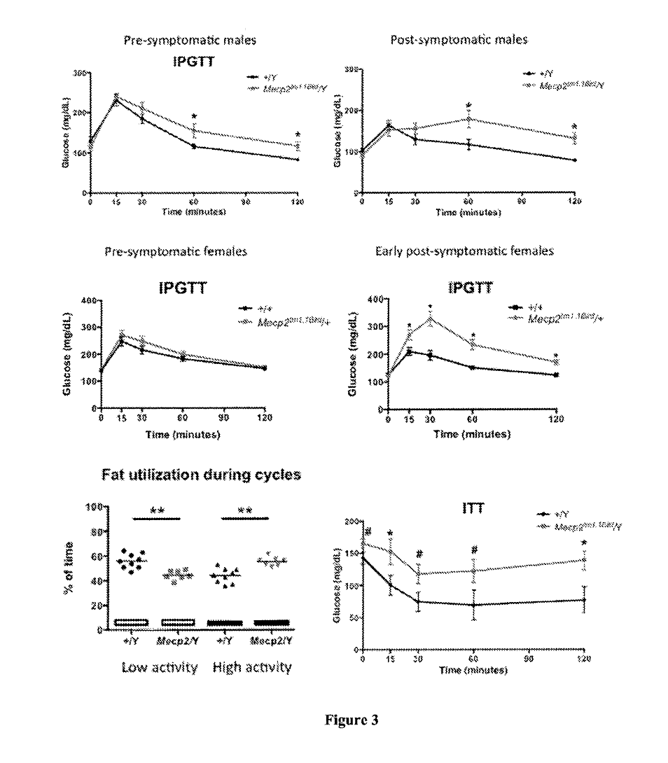

Glucose metabolism is inextricably linked to cholesterol and lipid metabolism through the action of a protein called 5′ AMP-activated protein kinase (AMPK), which acts as a master regulator of lipid, cholesterol, glucose, and protein metabolism, shunting small molecule precursors and energy from one activity to another. The FDA-approved drug metformin activates AMPK and is used to treat type 2 diabetes; its primary role in this case is inhibiting liver glucose production, but it has also been shown to prevent common cholesterol-related cardiac complications in diabetic patients. As demonstrated herein (see FIGS. 2 and 3), Mecp2 mutant mice display both cholesterol dysregulation and dysregulation of glucose and insulin metabolism similar to type 2 diabetes. Therefore, in accordance with some embodiments of the present invention, metformin and other related higuanide-class drugs may be useful agents in the treatment of Rett Syndrome and/or other MECP2-associated diseases, disorders, and conditions. In some embodiments, a glucose metabolism modulator such as 2,4-dinitrophenol-methyl ether (DNPME or DNP ME) may also be useful in treating one or more symptoms of Mecp2 related dysfunction.

Thus, in some embodiments, the present invention provides methods of treating a MECP2-associated disease, disorder, or condition (e.g., Rett Syndrome), which methods include a step of administering a glucose metabolism modulator to a subject suffering from or susceptible to the MECP2-associated disease, disorder, or condition. In some embodiments, the glucose metabolism modulator is selected from: a biguanide drug, 2,4-dinitrophenol-methyl ether (DNP-ME), 2,4-dinitrophenol-ethyl ether (DNP-EE), 2,4-dinitrophenol-vinyl ether (DNP-VE), derivatives of such compounds, and/or combinations thereof. In some embodiments, the biguanide drug is selected from: metformin, proguanil, chlorproguanil.

### SREBP Modulators

SREBPs regulate cholesterol and lipid metabolism upstream of HMG-CoA reductase, but downstream of AMPK. At least one indirect SREBP inhibitor, fatostatin, has been shown to effectively prevent and treat obesity, hypercholesterolemia, and hyperglycemia in mice and rats. In some embodiments, fatostatin or another indirect SREBP inhibitor is useful as a metabolic modulator as described herein. In some embodiments, one or more agents with a more direct mechanism of action to inhibit SREBPs are useful as metabolic modulators as described herein. For example, in some embodiments, fatostatin, SREBP1, SREBP2, and/or one or more nonspecific SREBP inhibitors is utilized to improve behavioral and/or metabolic symptoms in individuals suffering from or susceptible to a MECP2-associated disease, disorder or condition such as Rett Syndrome.

### PPARG Modulators

According to some embodiments of the invention, agents that target peroxisome proliferator-activated receptor gamma (PPARG) may be considered metabolic modulators for use as described herein. For example, agents that have been approved by the Food and Drug Administration (FDA) for the treatment of type 2 diabetes, some of which are PPARG activators, are useful in the treatment of a MECP2-associated disease, disorder or condition. In particular, the thiazolidinediones may be useful in the treatment of a MECP2-associated disease, disorder or condition. Thiazolidinediones are used in the treatment of type 2 diabetes because they effectively lower blood glucose levels without increasing pancreatic insulin secretion, but have also been shown to decrease fatty acid, LDL-cholesterol, and triglyceride production.

### Combinations

In some embodiments of the present invention, metabolic pathway modulators as described herein are utilized in combination with each other and/or with one or more other agents or therapeutic modalities that treats one or more symptoms of an MECP2-associated disease, disorder or condition, and/or that reduces incidence, frequency, and/or intensity of one or more undesirable side effects of therapy.

The phrase “in combination”, as used herein, refers to agents or modalities that are administered concurrently with, prior to, or subsequent to, one or more other desired therapeutics such that the subject is simultaneously exposed to both (or all) agents or modalities. Each of the two or more agents or modalities may be administered according to a different schedule; it is not required that individual doses of different agents be administered at the same time, or in the same composition. Rather, so long as both (or more) agents are present in the subject's body simultaneously for some period of time, they are considered to be administered “in combination”.

It is common for cholesterol lowering drugs with differing mechanisms of action to be used in combination, as is the case with statins and ezetimibe. Furthermore, because the product of HMG Co-A reductase, which statins inhibit, is required for multiple biological pathways, not just cholesterol production, statins are commonly given with supplements, such as mevalonate, to prevent unwanted effects caused by downregulating pathways that were not the desired target.

In many embodiments of the present invention, treatment of a MECP2-associated diseases, disorder, or condition may involve or require a combination of two or more metabolic pathway modulators or other agents or modalities as described herein. One example would he metformin combined with a PPARG agonist or with statins. Alternatively or additionally, in many embodiments, patients suffering from or susceptible to one or more MECP2-associated diseases, disorders or conditions (e.g., Rett Syndrome) are treated with agents or other therapeutic modalities for addressing common comorbiditics, such as epilepsy and hyperactivity. Indeed, as the primary benefit of addressing cholesterol and lipid dyrsregulation in Rett Syndrome has been improvement of metabolic profile and motor symptoms, it is expected in many embodiments that metabolic pathway modulator therapy as described herein will not replace current symptom-specific treatments, but rather will work in conjunction with them.

### Pharmaceutical Compositions

Provided agents and modalities that modulate lipid and/or cholesterol metabolism as described herein may be utilized in the context of a pharmaceutical composition. In general, a utilized pharmaceutical composition comprises at least one active agent and at least one pharmaceutically acceptable carrier or excipient. Such pharmaceutical compositions may optionally comprise and/or be administered in combination with one or more additional therapeutically active substances. In some embodiments, provided pharmaceutical compositions are useful in medicine. In some embodiments, provided pharmaceutical compositions arc useful as prophylactic agents. In some embodiments, provided pharmaceutical compositions are useful in therapeutic applications. In some embodiments, pharmaceutical compositions are formulated for administration to humans.

For example, in some embodiments, pharmaceutical compositions provided here may be provided in a sterile injectable form (e.g., a form that is suitable for subcutaneous injection or intravenous infusion) and/or other liquid dosage form that is suitable for injection. In some embodiments, pharmaceutical compositions are provided as powders (e.g., lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions comprise one or more pharmaceutically acceptable excipients (e.g., preservative, inert diluent, dispersing agent, surface active agent and/or emulsifier, buffering agent, etc.). In some embodiments, appropriate excipients for use in provided pharmaceutical compositions may, for example, include one or more pharmaceutically acceptable solvents, dispersion media, granulating media, diluents, or other liquid vehicles, dispersion or suspension aids, surface active agents and/or emulsifiers, isotonic agents, thickening or emulsifying agents, preservatives, solid binders, lubricants, disintegrating agents, binding agents, preservatives, buffering agents and the like, as suited to the particular dosage form desired. Alternatively or additionally, pharmaceutically acceptable excipients such as cocoa butter and/or suppository waxes, coloring agents, coating agents, sweetening, flavoring, and/or perfuming agents can be utilized.

In some embodiments, an appropriate excipient is at least 95%, at least 96%, at least 97%, at least 98%, at least 99%, or 100% pure. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or other International Pharmacopoeia.

In some embodiments, pharmaceutical compositions comprise one or more preservatives. In some embodiments, pharmaceutical compositions comprise no preservative.

In some embodiments, pharmaceutical compositions are provided in a form that can be refrigerated and/or frozen. In some embodiments, pharmaceutical compositions are provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of compositions for longer than the specified time results in degradation of active agents.

In some embodiments, liquid dosage forms (e.g., for oral and/or parenteral administration) include, but are not limited to, emulsions, microemulsions, solutions, suspensions, syrups, and/or elixirs. In addition to provided soluble lipidated ligand agents, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such a CREMOPHOR®, alcohols, oils, modified oils, glycols, polysorbates, cyclodextrins, polymers, and/or combinations thereof. Liquid dosage forms and/or reconstituted solutions may comprise particulate matter and/or discoloration prior to administration. In some embodiments, a solution should not be used if discolored or cloudy and/or if particulate matter remains after filtration.

In some embodiments, injectable preparations, for example, sterile aqueous or oleaginous suspensions, may be formulated according to known methods using suitable dispersing agents, wetting agents, and/or suspending agents. Sterile liquid preparations may be, for example, solutions, suspensions, and/or emulsions in nontoxic parenterally acceptable diluents and/or solvents, for example, as a solution in 1,3-butanediol. Among the acceptable vehicles and solvents that may be employed, for example, are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of liquid formulations.

Liquid formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, one or more strategies may be utilized prolong and/or delay the effect of a provided soluble lipidated ligand agent after delivery.

In some embodiments, solid dosage forms (e.g., for oral administration) include capsules, tablets, pills, powders, and/or granules. In such solid dosage forms, the provided soluble lipidated ligand agent(s) may be mixed with at least one inert, pharmaceutically acceptable excipient such as sodium citrate or dicalcium phosphate and/or fillers or extenders (e.g., starches such as maize starch, wheat starch, rice starch, potato starch;sugars such as lactose, sucrose, glucose, mannitol, sorbitol, and silicic acid), binders (e.g., carboxymethylcellulose, alginates, gelatin, polyvinylpyrrolidinone, sucrose, and acacia), humectants (e.g., glycerol), disintegrating agents (e.g., agar, Explotab, sodium starch glycolate, Amberlite, sodium carboxymethylcellulose, ultramylopectin, orange peel, natural sponge, bentonite, calcium carbonate, potato starch, tapioca starch, alginic acid, certain silicates, one or more insoluble cationic exchange resins, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

In some embodiments, solid compositions of a similar type may be employed as fillers in soft and/or hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

Exemplary enteric coatings include, but are not limited to, one or more of the following: cellulose acetate phthalate; methyl acrylate-methacrylic acid copolymers; cellulose acetate succinate; hydroxy propyl methyl cellulose phthalate; hydroxy propyl methyl cellulose acetate succinate (hypromellose acetate succinate); HP55; polyvinyl acetate phthalate (PVAP); Eudragit L30D; Eudragit L; Eudragit S; Aquateric; methyl methacrylate-methacrylic acid copolymers; methacrylic acid copolymers, cellulose acetate (and its succinate and phthalate version); styrol maleic acid co-polymers; polymethacrylic acid/acrylic acid copolymer; hydroxyethyl ethyl cellulose phthalate; hydroxypropyl methyl cellulose acetate succinate; cellulose acetate tetrahydrophtalate; acrylic resin; shellac, and combinations thereof. In some embodiments, an enteric coating is substantially impermeable to at least pH 5.0.

In some embodiments, solid dosage forms may optionally comprise opacifying agents and can be of a composition that they release the provided soluble lipidated ligand agent(s) only, or preferentially, in a certain part of the intestinal tract (e.g., the duodenum, the jejunum, or the ileum), optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In some embodiments, the present invention provides compositions for topical and/or transdermal delivery, e.g., as a cream, liniment, ointment, oil, foam, spray, lotion, liquid, powder, thickening lotion, or gel. Particular exemplary such formulations may be prepared, for example, as products such as skin softeners, nutritional lotion type emulsions, cleansing lotions, cleansing creams, skin milks, emollient lotions, massage creams, emollient creams, make-up bases, lipsticks, facial packs or facial gels, cleaner formulations such as shampoos, rinses, body cleansers, hair-tonics, or soaps, or dermatological compositions such as lotions, ointments, gels, creams, liniments, patches, deodorants, or sprays.

Pharmaceutical compositions described herein may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to a dose which would be administered to a subject and/or a convenient fraction of such a dose such as, for example, one-half or one-third of such a dose.

Relative amounts of active ingredient, pharmaceutically acceptable excipient, and/or any additional ingredients in a pharmaceutical composition in accordance with the invention may vary, depending upon the identity, size, and/or condition of the subject treated and/or depending upon the route by which the composition is to be administered. By way of example, the composition may comprise between 0.1% and 100% (w/w) active ingredient.

Pharmaceutical compositions of the present invention may additionally comprise one or more solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

### Routes of Administration

In some embodiments, provided agents may be formulated for any appropriate route of delivery. In some embodiments, provided agents may be formulated for a route of delivery, including, but not limited to, intramuscular (IM), intravenous (IV), intraperitoneal (IP), subcutaneous (SQ), bronchial instillation, and/or inhalation; buccal, enteral, interdermal, intra-arterial (IA), intragastric (IG), intramedullary, intranasal, intrathecal, intratracheal instillation (by), intraventricular, intra-articular, mucosal, nasal spray, and/or aerosol, oral (PO), as an oral spray, rectal (PR), sublingual; topical and/or transdermal (e.g., by lotions, creams, liniments, ointments, powders, gels, drops, etc.), transdermal, vaginal, vitreal, and/or through a portal vein catheter; and/or combinations thereof. In some embodiments, the present invention provides methods of administration of provided agents via direct injection (e.g., into a specific tissue such as the brain). In some embodiments, the present invention provides methods of administration of provided agents via intravenous administration. In some embodiments, the present invention provides methods of administration of provided agents via oral administration. In some embodiments, the present invention provides methods of administration of provided agents via subcutaneous administration.

In some embodiments, an agent is administered in a tissue-specific manner In some embodiments, an agent is administered directly to the brain.

### Dosing

Any of a variety of doses are contemplated as compatible with various embodiments. It is contemplated that a proper dose in a particular application will be determined in accordance with sound medical judgment. By choosing among various agents and weighing factors such as potency, relative bioavailability, patient body weight, severity of adverse side-effects and preferred mode of administration, an effective prophylactic or therapeutic treatment regimen can be planned which does not cause substantial unwanted toxicity and yet is effective to treat a particular subject. The effective amount for any particular application can vary depending on such factors as the particular agent of the invention being administered, the size of the subject, and/or the severity of the disease or condition.

In some embodiments, it is preferred that a maximum dose be used, that is, the highest safe dose according to sound medical judgment. Multiple doses per day arc contemplated as useful in some embodiments to achieve appropriate systemic levels of a provided agent. Appropriate systemic levels may be determined by, for example, measurement of a subject's peak or sustained plasma level of the agent.

In some embodiments, daily doses of agents will be, for human subjects, from about 0.01 mg/kg per day to 1,000 mg/kg per day (e.g., about 1.5-30 mg/kg/day). Specific doses may be adjusted appropriately to achieve desired drug levels, local or systemic, depending upon the mode of administration. In some embodiments, multiple doses per day are contemplated to achieve appropriate systemic levels of agents. Provided agents may be formulated into a controlled and/or sustained release form.

In some embodiments, an agent is or comprises a drug approved by the FDA. In some embodiments, such agents are administered according to the FDA-approved dosing regimen for the drug. In some embodiments, such agents are administered according a dosing regimen that is different from the FDA-approved dosing regimen. In some embodiments, such agents are administered at one or more of a lower dose, less frequent dosing schedule, and/or fewer total doses as compared to the FDA-approved dosing regimen. In some embodiments, such agents are administered at a dose between 10× less than the FDA-approved dose and 10× more than the FDA approved dose.

Identification and/or Characterization of Metabolic Pathway Modulators

In some embodiments, the present invention provides systems for identifying and/or characterizing useful metabolic pathway modulators for use in treating disease, disorders, or conditions, e.g., associated with aberrant MECP2. In particular, the present invention provides systems for identification and/or characterization of modulators of lipid and/or cholesterol metabolism (e.g., biosynthesis) pathways, and particularly of lipid and/or cholesterol pathways in the brain.

In some embodiments, the present invention identifies and/or characterizes agents based on their effects on one or more particular components of a metabolic pathway, and particularly of a lipid and/or cholesterol metabolism (e.g., biosynthesis) pathway, most particularly of a lipid and/or cholesterol metabolism (e.g., biosynthesis) pathways in the brain. In some particular embodiments, the present invention identifies and/or characterizes agents based on their effects on squalene epoxidase (Sqle), also known as squalene monooxygenase, or other components of pathways in which Sqle participates. In some particular embodiments, the present invention identifies and/or characterizes agents based on their effects on presence, level, activity, and/or form of 24S-OHC.

In some embodiments, the present invention further provides methods of assessing the effect(s) of one or more agents or modalities on lipid and/or cholesterol metabolism by assessing the level of 24SOHC in a subject exposed to one or more such agent or modality. In some embodiments, the assessment of 24SOHC is made using a blood sample from a subject. In some embodiments, the assessment of 24SOHC is made using a biological sample other than a blood sample (e.g., cerebrospinal fluid). The detection/assessment of 24S-OHC may be via any suitable methodology including, but not limited to: antibody-based detection (e.g., ELISA), radiolabeling, ligand-binding assays, mass spectrometry, high pressure liquid chromatography, and/or enzyme activity assays.

In some embodiments, the present invention provides systems for identifying and/or characterizing such agents by contacting them with a system that comprises one or more such metabolic pathway components, and assessing their impact on presence, level, activity, and/or form of one or more indicators (e.g., components, products, and/or markers of the relevant pathway(s)). In some embodiments, a provided system comprises a complete and/or active metabolic pathway (e.g., a lipid or cholesterol biosynthesis pathway).

In some embodiments, provided identification and/or characterization systems comprise one or more cells, tissues, and/or organisms. In some embodiments, such systems are or comprise mouse cells, tissues, and/or organisms. In some embodiments, such systems are or comprise one or more mouse cells, tissues, and/or organisms that show reduced expression and/or activity of MECP2 (e.g., as a result of genetic mutation and/or chemical alteration).

In accordance with specific embodiments of the present invention, certain phenotyping (symptom) assessments have been established to determine the extent to which potential modulator agents alter cognitive ability, motor function, and/or physiological parameters. In some embodiments, a model organism (e.g., an engineered mouse) is utilized. In some embodiments, a MECP2-deficient mouse is utilized. In some embodiments, results are compared with a reference mouse in which MECP2 deficiency is compensated, e.g., by mutation of Sqle.

In some embodiments, effects of potential modulators are assessed in or on female cells (e.g., in female mice). A recent publication shows that heterozygous Mecp2^{tm1.1Bird}/+ females perform well on certain assays that will allow us to assess the degree of rescue in females as well (Samaco et al. Hum Mol Genet 22: 96-109, 2013).

In some embodiments, effects of potential modulators are assessed using behavior assays (e.g., in mice) for acoustic startle response (ASR), pre-pulse inhibition of startle response (PPI), open field activity, three chamber social interaction and/or combinations thereof. In some embodiments, effects of potential modulators on weight gain and overall health are assessed (e.g., periodically such as daily, weekly, biweekly, or monthly).

In some embodiments, effects of potential modulators are assessed with respect to breathing anomalies. For example, methacholine challenge analysis in Buxco whole body plethysmography chambers carried out on male and female mice at seven weeks accurately assesses breathing anomalies, and this defect is also ameliorated by the use of statin drugs.

The Table below presents a representative time course and order of exemplary that can be utilized in accordance with the present invention to identify and/or characterize agents of interest.

| | TEST | | | | | |
|---|---|---|---|---|---|---|
| | MALES MECP2−/Y | | | FEMALES MECP2−/+ | | |
| | Weekly body weight | | | | | |
| | + | | | + | | |
| | 4 weeks | 8 weeks | 12 weeks | 8 weeks | 12 weeks | 20 weeks |
| Subjective health | + | + | + | + | + | + |
| assessment | | | | | | |
| Open field activity | − | + | − | − | − | + |
| Rotarod | − | + | − | − | + | + |
| Acoustic startle | − | + | − | − | − | + |
| response | | | | | | |
| PPI of ASR | − | + | − | − | − | + |
| DEXA | − | − | + | − | − | + |
| Social activity | − | + | − | − | − | + |
| Breathing + | − | + | − | + | − | − |
| challenge* | | | | | | |
| Clinical chemistry | − | − | + | − | − | + |
| Brain and Liver lipid | − | − | + | − | − | + |
| panel | | | | | | |

In some embodiments, serum chemistries, and/or brain and liver lipids are assessed. Particularly if evidence for lipid modulation is evident, additional assays for metabolic status, including intraperitoneal glucose tolerance tests (IPGTT), insulin tolerance (ITT) and calorimetry may be carried out.

In some embodiments, effect(s) of potential modulators are assessed via one or more of a: behavioral test, cognitive test, motor function test, test of one or more physiological parameters, and combinations thereof.

In some embodiments, a relevant behavioral test is selected from: acoustic startle response test, pre-pulse inhibition of startle response test, open field activity test, three chamber social interaction test, Home Cage Activity test, and/or combinations thereof.

In some embodiments, a relevant motor function test is selected from: breathing challenge, rotarod test, open field locomotor activity test, DigiGait System to monitor gait (from Mouse Specifics), and combinations thereof.

In some embodiments, a test of one or more physiological parameters is selected from: dual X-ray absorptiometry (DEXA) test, whole body plethysmography breathing test with methacholine challenge, glucose tolerance test, insulin tolerance test, serum cholesterol test, calorimetry test, and combinations thereof.

Unless otherwise stated, all publications, patents, and patent applications mentioned herein are hereby incorporated by reference in their entirety as if each individual publication, patent, or patent application was specifically and individually indicated to be incorporated by reference. In case of a conflict, the present application, including any definitions herein, will control.

### Exemplification

Materials and Methods: The below Materials and Methods were utilized in the Examples that follow, unless otherwise stated.

### Animal Experiments

All experiments carried out on animals were approved by the Institutional Animal Care and Use Committee at Baylor College of Medicine in accordance with guidelines established by the National Institutes of Health. Drug treatment experiments were blinded in regards to genotype and treatment group. Unless otherwise described, all animal experiments were performed according the conditions and protocols detailed in Buchovecky et al., A genetic suppressor screen in Mecp2 mice implicates cholesterol metabolism in Rett Syndrome, 2013, Nat. Genet., 45(9): 1013-1020, and summarized here. Briefly, all animals treated with statins were 129.Mecp2^{tm1.1Bird}/Y or 129.Mecp2^{tm1.1Bird}/+ and their sex-matched wildtype littermates. Unless otherwise specified, mice were housed in plastic Lab Products cages with corncob bedding in rooms alternating 13-hr and 11-hr periods of light and dark were provided acidified water, and a Harlan Teklad 2920X diet ad libitum (19.1% protein, 6.5% fat; 0% cholesterol). Subsequent to the pilot study with fluvastatin, that included 6 Mecp2-null mice per treatment group, all behavioral assessments were performed with the experimenter blinded to treatment group. For other statin treatments, all behavioral assessments were performed with the experimenter blinded to treatment group. All chemical assays were performed blinded to genotype and treatment group. Unless otherwise specified, tissue and blood collection took place within a two hour afternoon window following a 4-6 hour fast. Brain analyses were performed on the subcortical region, which contains the corpus callosum, striatum, thalamus, hypothalamus, and hippocampus.

### Q-PCR Analysis

Brain RNA was isolated using RNAeasy Lipid Tissue Mini Kit (Qiagen) and liver RNA using Trizol (Invitrogen), according to manufacturer's instructions. Liver RNA was treated with 1 IU DNAse (Ambion Inc.) at 37° C. for 1 h per manufacturer's instructions. First strand complementary DNA (cDNA) was synthesized from 5000 ng of total RNA using SuperScript III First Strand Synthesis System (Invitrogen) per manufacturer's instructions. RT-PCR was performed in triplicate for each sample on an ABT 7900 (Applied Biosystems CA, USA). Gene primers for QRT-PCR were designed against published mRNA sequences using Primer3 software and synthesized by Integrated DNA Technologies (Iowa, USA). Primer sequences will we provided upon request. QRT-PCR was performed in triplicate on an ABI 7900 (Applied Biosystems CA, USA). Reactions contained cDNA from 10 ng total RNA, 0.1 μL forward and reverse primers, 5 μPower SYBR®Green Master Mix, and water to a final volume of 10 μL. PCR conditions: 95° C. for 10 min, 40 cycles of 95° C. for 15 sec, 60° C. for 60 sec. Single product amplification was confirmed by disassociation curves and agarose gel electrophoresis. Gene expression was normalized to an RpL19 (L19) internal loading control, and analyzed using the 2^{-(ΔΔCT)} method expressed either as raw 2^{-(ΔΔT)} or as Mecp2/Y expression relative to WT.

### Tandem Mass Spectrometry Analysis of Mouse Brain and Liver Samples

Cholesterol intermediates were measured after extraction from tissue from mice treated the same as above by tandem mass spectrometry following a previously published protocol (McDonald et al. J Lipid Res 53: 1399-1409, 2012).

In vivo Cholesterol Synthesis Analysis

For the in vivo cholesterol synthesis study, samples were obtained from mice in a fed state the late dark phase of a 12-hour on/off light dark cycle. These mice were adapted to individual housing and a Harlan Teklad 7001 rodent chow (low cholesterol 0.02% w/w, low fat 4% w/w) starting at P38 prior to analysis. The age of mice at sampling was P54-56. Cholesterol synthesis was assessed from saponified tissue after the incorporation of 100 mCi ³H₂O after intraperitoneal injection as published (Xie et al. J Lipid Res 44: 1780-1789, 2003).

### Drug Administration

Fluvastatin (Selleckchem) was dissolved in sterile ultrapure water such that the desired dose for a 20 g mouse was given in 100 ul and administered subcutaneously. Male mice were given a single 3 mg/kg weekly dose at five, six and seven weeks, then were given 3× weekly (M,W,F) 3 mg/kg doses beginning at 8 weeks of age. Female mice also received 3 mg/kg doses, but were treated only once per week, beginning at 6 weeks of age. Lovastatin (Tocris Bioscience) preparation required activation in ethanol followed by adjustment to pH7.2, per product information guidelines. The activated stock solution was diluted with ethanol to 20× the injected dose and kept at −20° C. for up to one month. The day of injection a 1× working solution was prepared by diluting the stock solution in sterile saline such that the desired dose for a 20 g mouse was given in 100 ul. Male mice were injected subcutaneously with a twice-weekly 1.5 mg/kg dose, beginning at five weeks of age.

### Accelerating Rotarod Task

An aspect of motor performance was measured using the accelerating rotating rod (rotarod) (Stoelting). At 8 weeks (males) or 12 weeks (females), mice were placed on a grooved rod, rotating at a speed of four revolutions per minute. Over the course of a five-minute trial, the revolution rate increases steadily to a maximum of forty revolutions per minute. The time each mouse is able to stay on the rod is recorded for eight trials, four each over two consecutive days, with a minimum of thirty-minutes between trials. A trial ends when the mouse falls off the rod, spins with the rod for two consecutive revolutions, or successfully completes five minutes.

### Open Field Locomotor Activity

Open field locomotor activity was assessed using Versamax Animal Activity Monitors. Recordings were taken in a secluded room with dim light (20-25 lux) and artificial white noise (55-60 dB). Each mouse was placed in the center of the open field chamber and activity was recorded for 30 minutes. These measurements were taken at 10 weeks (males) or 5 months (females); 24 hours after last injection.

### Pre-Pulse Inhibition Assay

Pre-pulse inhibition was measured using SR-Lab Startle Chambers (San Diego Instruments). Over the course of a 15 minute trial, mice are exposed to a random series of acoustic pulses or pairs of pulses designed to elicit an acoustic startle response (50 decibels), as well as to mitigate that response when the decibel (dB) pulse is preceeded by a softer pulse (4, 8, or 12 decibels). Each mouse is exposed to every possible configuration 6 times throughout the course of the trial.

### Lipid Measurements

Prior to gas-liquid chromatography, lipids were isolated from tissue using CHCl₃:CH₃OH extraction, followed by drying of the organic phase under N₂ pressure. Lipids were then redissolved in 500 μl of PBS-5% Triton X100. Serum cholesterol was measured on a Cobas Mira clinical chemistry analyzer.

### Statistical Analysis

Survival curves were compared using SPSS by Kaplan-Meier analysis followed by log rank comparison. Statistical comparisons between two groups (wild type compared to Mecp2 mutant) were performed in GraphPad Prism 5 using an unpaired, two-tailed student's t-test; equal variances were not assumed, as the Mecp2 mutant group typically showed increased variability compared to wild type. Statistical tests requiring multiple comparisons were analyzed in SPSS. Excepting rotarod data, comparisons between multiple groups were analyzed by one-way ANOVA, sphericity not assumed; the Bonferroni adjustment was applied when comparing more than two genotypes, the Dunnett post hoc test was used to compare statin treated groups with the vehicle control. Rotarod data was analyzed using repeated measures ANOVA.

### Metabolic Assays

Intraperitoneal glucose tolerance tests (IPGTT) were performed at 4 and 8 weeks of age in males, and at 12 and 24 weeks of age in females. After a four-hour fasting period, animals were lightly anesthetized with isoflurane. A small tail amputation was made and blood was collected for a 0 time point. Blood glucose was sampled using the AlphaTRAK Blood Glucose Monitoring System (Abbott Laboratories, IL, USA) prior to treatment, and then at 15, 30, 60 and 120 minutes (t=15, t=30, t=60 and t=120) after glucose injection, per manufacturer's instructions. Mice were each intraperitoneally injected with glucose (2 g/kg bodyweight).

Intraperitoneal insulin tolerance tests (IPITT) were performed by The Diabetes and Endocrinology Research Center at BCM (P30 DK079638). Briefly, following a 4 hour fast, mice were administered insulin (0.75 milliunit/g bodyweight) and glucose levels were obtained at 0, 15, 30, 60 and 120 minutes.

Respiratory metabolic function was assessed using the Oxymax Deluxe System indirect calorimeter equal flow eight chamber system (Columbus Instruments, Columbus, Ohio) and analyzed using Oxymax Windows V3.32 Software. Mice were housed individually in calorimetry cages for a period of 24 hours beginning with a 3 hour light-phase acclimatization period (12:00-15:00), followed consecutively by a 4 hour light-phase (15:00-19:00), a 12 hour dark-phase (19:00-07:00), and a minimum five hour light-phase (07:00-12:00). Cumulative food intake, temperature, volume of oxygen consumed and volume of carbon dioxide produced were measured. From these data the respiratory exchange ratio and energy expenditure were derived.

### EXAMPLE 1

### Identification and Characterization of Gene Targets for RTT

The present Example describes a genetic suppressor screen that was performed in Mecp2 null mice (“Mecp2 mice”), a well-accepted mouse model of Rett Syndrome, and identified certain new targets for RTT therapy.

Identifying “suppressor” genes (i.e., genes that when mutated ameliorate or prevent worsening of the symptoms of a disease, disorder or condition associated with a defect in a particular “disease” gene) helps to focus efforts towards understanding of how the disease gene functions and how the relevant disease, disorder or condition ensues upon loss of such function. Further, such suppressor genes can reveal new pathways that can be targeted to reverse or prevent progression of symptoms.

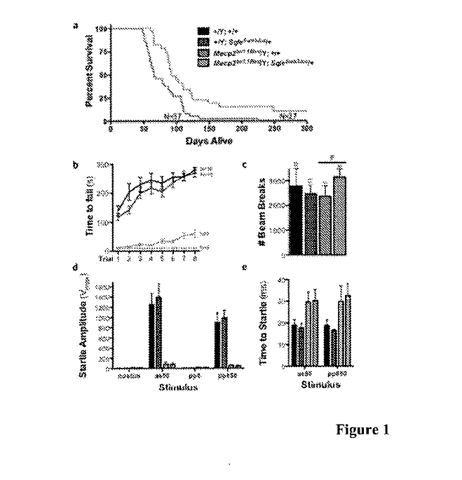

The present Example demonstrates (see FIG. 1) identification of a nonsense (STOP) suppressor mutation in squalene epoxidase (Sqle), also known as squalene monooxygenase, which suppresses symptoms of RTT in Mecp2 mice (specifically, in Mecp2^{tm1.1Bird} mice, obtained from The Jackson Laboratory). Sqle encodes a rate-limiting enzyme in cholesterol synthesis; the present Example therefore identifies the cholesterol biosynthesis pathway as an appropriate target for RTT therapy.

In particular, the present Example describes a “modifier” genetic screen in which Mecp2 null mice were mutagenized with a powerful mutagen that alters many genes in the genome simultaneously, it being expected that only a few mutations will alter the phenotype associated with the extant MECP2 disruption. Specifically, wild type C57BL/6J male mice were mutagenized with the chemical supermutagen N-ethyl-N-nitrosourea (ENU), which is known to provide an appropriate level of mutagenic power (Justice Nat Rev Genet 1: 109-115, 2000). ENU typically causes point mutations. Possible genetic outcomes of mutagenesis with ENU include loss of function, gain of function, super-active, and partially active coding region mutations, as well as non-coding RNA and regulatory mutations. Mutations were identified by sequencing.

The mouse genetic screen utilized a random chemical mutagenesis screen, in which symptom rescue may be conferred by any mutation in an unknown gene. Subsequent identification of the gene involved inheritance and sequencing studies. ENU-treated C57BL/6J males were bred to female 129.Mecp2^{tm1.1Brd}/+ mice (the strain is maintained as a loss of function line congenic on 129S6/SvEvTac). Male offspring in the first generation (G₁), asymptomatic at weaning, were genotyped for presence of mutant Mecp2, and examined for suppression of disease phenotypes by a dominant mutation, which would rescue neurological symptoms and perhaps result in longevity. Six-hundred and seventy-nine males that carry the null Mecp2 mutant allele were screened for rescue of neurological defects and increased survival. Among five suppressors, the screen identified the Sqle mutation, which suppresses symptoms by modifying cholesterol metabolism in MECP2 mutant mice. An additional 3700 gametes will be screened; symptom amelioration in the line is confirmed by breeding and assessment of up to 300 offspring per mutant line.

In a preliminary screen, five suppressors of MECP2 symptoms in mice were identified. The data implicated certain metabolic pathways in RTT, and suggested that drugs developed to treat metabolic disorders were likely to improve symptoms of RTT and/or of other disorders associated with MECP2.

One particular suppressor mutation of interest, Sum3, was determined to be a stop codon mutation in squalene epoxidase (Sqle) (Buchovecky et al. Nature Genetics, in press 2013). Sqle catalyzes the first oxygenation reaction in the committed production of cholesterol. Cholesterol levels feedback to influence the activity of both SQLE and HMG Co-A reductase (HMGCR) by independent mechanisms, both of which are critical for cholesterol homeostasis (Yamamoto and Bloch J Biol Chem 245: 1670-1674, 2008; Gill et al. Cell Metab 13: 260-273, 2011). Sqle is widely expressed and its primary product, 2,3-oxidosqualene, is a transient intermediate that is immediately cyclized to lanosterol by lanosterol synthase (Lss) (Cory et al J Am Chem Soc 88: 4750-4751 (1966). SQLE is well conserved throughout evolution; the mouse and human proteins are 84% identical.

Elevated cholesterol biosynthesis in the brain of Mecp2 null mutants could contribute to neurologic dysfunction: the stop codon mutation likely down-regulates the pathway to confer rescue. The data provided herein showed for the first time that cholesterol metabolism is disrupted in the brain and liver of mouse models of Rett Syndrome. Cholesterol metabolism was assessed by Q-RT-PCR, gas-liquid chromatography, mass spectrometry, and quantitation of cholesterol synthesis (Xie et al. 2003) in the brains and livers of two Mecp2 null alleles: Mecp2^{tm1.1Bird} and Mecp2^{tm1.1Jac}. When Mecp2^{tm1.1Bird}/Y mice display minimal symptoms, Cyp46a1 expression is already increased (38-fold over wild type; p>0.05) in the null brain, indicating a heightened need for cholesterol turnover in early stages of disease.

Strikingly, cholesterol synthesis was decreased in the brain of moderately symptomatic mice by 23% per gram of tissue, which is unusual when considering the presence of a variety of cell types in many states of activity. Interestingly, brain cholesterol concentration per gram was slightly increased in the face of lower de novo synthesis. Cyp46a1 −/− is the only other mouse mutant that exhibits decreased brain cholesterol synthesis, yet it has no change in cholesterol concentration per gram of tissue and no change in brain mass (Lund et al. J Biol Chem 278: 22980-22988, 2003). The analysis of Cyp46a1 −/− demonstrated the importance of brain cholesterol turnover for neurological function. Cholesterol turnover may be required to produce geranylgeraniol, a product of HMGCR upstream of SQLE that is essential for learning and synaptic plasticity, and may be important for the interaction between neurons and astrocytes at the synapse. Therefore, the present disclosure establishes that dysregulation of cholesterol metabolism in neurons is potentially a major contributor to the development of symptoms in Mecp2 null males. Failure of cholesterol turnover may explain the contribution of glial-specific Mecp2 expression to the mitigation of RTT symptoms in a non-cell autonomous manner (Ballas et al. Nat Neurosci 12: 311-317 (2009).

Dyslipidemia is accompanied by a metabolic syndrome in male and female Mecp2 null mice. Metabolic and endocrine challenge experiments were carried out to determine if the Mecp2 null dyslipidemia phenotype led to metabolic disease. The basal glucose levels of 4-hour, 6-hour, and 16-hour fasted Mecp2 mice do not significantly differ from wild type controls, suggesting that Mecp2 mice are not hyperglycemic, and therefore are not diabetic. However, Mecp2 mice have decreased sensitivity to bolus administration of glucose during an intraperitoneal glucose tolerance test (IPGTT), indicating either an inherent incapability of the pancreas to produce insulin, or a decreased sensitivity of the tissues to the action of insulin. As early as four weeks of age, MECP2 null male mice displayed impaired glucose tolerance, as evident by a significant increase in the area under the curve (AUC) following glucose challenge, which was more pronounced at eight weeks of age (P=0.003; FIG. 3). MECP2 mice are less capable at clearing glucose from their blood in response to an exogenous bolus of insulin, indicating that they are insulin resistant. Therefore, the glucose tolerance observed in MECP2 mice likely stems from a decreased sensitivity to insulin action rather than pancreatic defects. In support of normal pancreatic function, MECP2 mice have comparable levels of ketone bodies in the blood following a 0-hour, 6-hour, and 24 hour fast. Lipid homeostasis is regulated in a diurnal manner based on feeding and activity behavior. During the day, when the nocturnal mouse is inactive and feeding less, genes involved in lipid synthesis and sequestration are repressed. This allows the pool of metabolic precursors in the liver to be shunted towards gluconeogenesis in order to maintain normoglycemia. At night, when mice are active and feeding, genes involved in the lipogenic pathways are expressed, promoting the storage of extraneous metabolites as triglycerides, without directly effecting the expression of gluconeogenic transcripts Energy expenditure was examined by indirect calorimetry. MECP2 null male mice used fat as an energy source in preference to glucose during periods of activity, suggesting that their ability to use glucose is impaired, and is a sign of metabolic syndrome.

### EXAMPLE 2

### Treatment of RTT-Like Symptoms in Mice with Cholesterol Biosynthesis Modulators

The present Example demonstrates that treatment of MECP2 mutant mice (which, as described herein, represent an established RTT model) with statins improves motor symptoms and increases longevity. The present Example further demonstrates that trait amelioration occurs by modifying the abnormal synthesis and deposition of lipids in the brain and liver of MECP2 male and female mice, and thereby establishes that such abnormal synthesis and deposition causes some or all of the metabolic defects associated with RTT. The present Example thus establishes that statin drugs can be used to alleviate the abnormal lipid deposition and improve motor symptoms in male and female mice. Those of ordinary skill in the art, reading the present specification, including this Example, will appreciate that it establishes the principle that compounds effective in treatment of certain metabolic disorders may be metabolic modulators for use as described herein. A potential patient population that may be aided by cholesterol lowering drugs are individuals with mutations in MECP2 or with mutations that alter dosage, function, or localization of those complexes which MECP2 recruits or anchors to the genome.

The present Example specifically demonstrates that cholesterol lowering drugs, including the statins, alleviate symptoms of MECP2 mutation in male and female mice. Tested compounds included: 1) fluvastatin; 2) simvastatin, 3) lovastatin and 4) atorvastatin. Drugs were administered in different doses to male and female mice to test their effects on symptom rescue: equivalent to, and 10-fold more than their published effective dosage in rodents. Drugs were administered sub-cutaneously to bypass liver metabolism. So far as we are aware, such drugs have not previously been used to treat Rett Syndrome, or any other disease, disorder or condition associated with mutation of Mecp2.

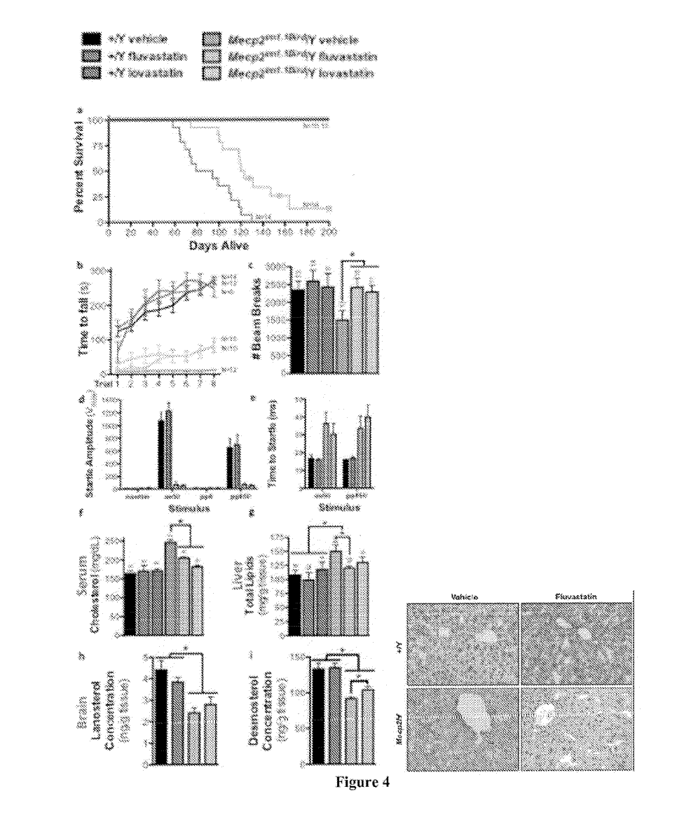

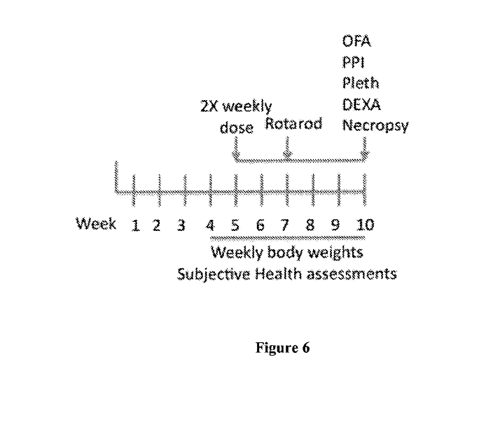

Consequences of trait amelioration were evaluated by body weight and body fat measurements, as well as assessments of neurological and breathing activity and general health (FIG. 6). As can be seen, statin drugs including fluvastatin, lovastatin, simvastatin and atorvastatin conferred varying degrees of rescue of motor traits and longevity, with fluvastatin and lovastatin conferring the best rescue in male mice (FIG. 4). Administration of statin drugs resulted in beneficial effects on brain cholesterol synthesis, as well as alleviation of the accumulation of lipids in the liver.

It is worth noting that various other drugs, including two that target squalene synthase ((E)-2-[2-fluoro-2-(quinuclidin-3-ylidene)ethoxy]-9H-carbazole monohydrochloride) or squalene epoxidase (1-(ethylsulfonyl)-2-piperidylethane), did not significantly improve symptoms. However, the effective doses and routes of administration of these drugs were less well established than the statin drugs, making it possible that newer compounds would also effectively lower cholesterol at the appropriate dosage. Since the defect in MECP2 mice affects both cholesterol and lipid storage, modulation of HMGCR, further upstream in the pathway, may be more effective. This suggests that the modifier pointed to a drug targetable pathway without the random mutation in Sqle conferring the best amelioration of traits.

Statin drugs improve motor symptoms by preventing lipid accumulation in the liver and by maintaining brain cholesterol synthesis. FDA-approved statin drugs provide a pharmacological means to down-regulate the cholesterol biosynthesis pathway by inhibiting HMGCR. In a preliminary trial, age-matched 129 Mecp2^{tm1.1Bird}/Y and +/Y littermates were treated with subcutaneous injections of fluvastatin. Treatment decreased scrum cholesterol, improved rotarod behavior and open field activity, and increased lifespan when compared with control mice receiving a sham dose. The statin drug did not rescue all health parameters commonly associated with mouse models of RTT, including acoustic startle response. However, it improved levels of cholesterol biosynthesis products toward wild type levels in the brain as assessed by mass spectrometry. These data support the idea that modulating the cholesterol biosynthesis pathway ameliorates motor symptoms of Mecp2 mutation in mice. Learning and memory tests for cognitive function were not included because they are dependent on motor behavior in mice and could be misinterpreted.

Mecp2^{tm1.1Bird} null males develop a severe metabolic disease that leads to hepatic steatosis, which likely plays a role in their untimely death. The amelioration of symptoms by statin drug administration influences both brain and systemic cholesterol homeostasis in Mecp2 null mice. Fluvastatin is not predicted to efficiently cross the BBB (Guillot et al., J Cardiovasc Pharmacol 21: 339-346, 1993); however, statin drugs can lower brain cholesterol synthesis through systemic effects on liver cholesterol metabolism (Cibickova L, J Clin Lipidol 5: 373-379, 2011). Lovastatin is more lipophilic, and crosses the BBB more efficiently.

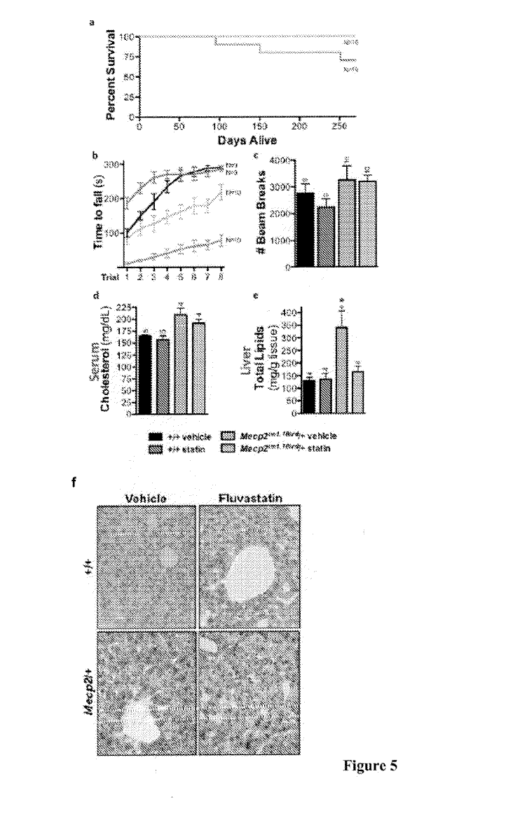

To provide more relevance to females affected with Rett Syndrome, we administered fluvastatin to female mice to determine the degree of rescue: females were treated 1× weekly from the age of six weeks to the age of 8 months. Similar to male mice, fluvastatin rescued the motor behavior, longevity and improved the fatty liver disease (FIG. 5). Notably, Mecp2/+ female mice develop a similar metabolic disease, albeit later in life than male Mecp2/Y mice, with a time of onset that correlates with increasing symptom severity.

In light of the positive data with statins, other lipid modulating drugs, including LXR inhibitors and metabolic modulators, are being evaluated in our drug testing protocol (FIG. 6) using the sub-Q or oral routes of administration to assess their ability to alleviate Rett Syndrome symptoms. Such drugs include: fatostatin, which modulates SREBP2 (a regulator of the cholesterol pathway), metformin, a commonly used metabolic modulator that activates AMPK, and SR9238 and bexarotene, LXR modulators.

### EXAMPLE 3

### RTT Patients Susceptible to Therapy with Metabolic Modulators

The present Example defines characteristics of individuals (e.g., Rett Syndrome patients) likely to benefit from therapy with metabolic modulators as described herein.

Specifically, the present Example demonstrates that Sqle is elevated in Mecp2 null male mice, and that the cholesterol biosynthesis pathway is perturbed in both brain and liver of Mecp2 null male mice (FIG. 2). Of note, elevated cholesterol, triglycerides and low density lipoproteins were a peripheral feature of disease in the mice, and may be a biomarker for patients that may respond to cholesterol-lowering drugs. Perturbation of the cholesterol metabolism pathway was not previously reported in Rett patients that carry mutations in MECP2, or in Mecp2 null mice. The present Example demonstrates that perturbed lipid metabolism leads to the development of fatty liver disease in the mice as well as a metabolic syndrome (FIGS. 2 and 3).

Statin drugs lower elevated peripheral cholesterol and triglycerides in one inbred strain of mice (129S6/SvEv) when the Mecp2 mutation is present, but in another strain, C57BL/6J, elevated cholesterol and triglycerides are not present with the Mecp2 mutation. This finding suggests that elevated serum lipids will be a biomarker in only a subset of patients.

Emerging evidence indicates that abnormalities in fatty acid metabolism may contribute to neurodevelopmental disorders such as autism and Rett Syndrome (Wiest et al. Fatty Acids 80: 221-227, 2009; Sticozzi et al. FEBS Letters doi.org/10.1016, 2013). The latter paper shows for the first time that total cholesterol, including LDL- and HDL-cholesterol are statistically elevated in Rett patients, suggesting that data provided herein, using the mouse model, is expected to translate into the human population. Also, 54% of RTT patients, correlating with those having the most severe MECP2 mutations, also have high lipid parameters that can be detected in the blood early in their diagnosis. The present Example teaches that patients with evidence for abnormal lipid parameters may be aided by the administration of drugs that regulate lipid metabolism, and that elevated serum cholesterol or LDL-cholesterol may serve as a biomarker for those patients that may respond to treatment with lipid modulating drugs.

The present Example therefore teaches that patients who display one or more symptoms of fatty liver disease, and/or who show elevated cholesterol triglycerides (e.g., elevated serum cholesterol) and low density lipoproteins in brain and/or liver tissues are candidates for therapy with metabolic modulators (e.g., statins) as described herein. Those skilled in the art will appreciate that any of a variety of methodologies may be utilized to detect such symptoms of fatty liver disease, and/or such elevated cholesterol triglycerides and low density lipoproteins in brain and/or liver tissues, and/or to detect proxies (i.e., correlated features or items) thereof.

### EXAMPLE 4

### Treatment of RTT-Like Symptoms in Mice with Statins Having Various Degrees of Lipophilicity

As shown above in Example 2, statins represent a previously unknown class of potential therapies for the treatment of Mecp2-related diseases, disorders, or conditions, such as Rett Syndrome (see FIG. 4). This Example extends those findings and provides insight that use of lipophilic statins may produce superior results, according to various embodiments of the present invention. In particular, fluvastatin may be particularly useful in treating Mecp2-related diseases, disorders or conditions.

In this Example, Fluvastatin, lovastatin, simvastatin, and atorvastatin were used. Fluvastatin is soluble in water and does not require activation prior to treatment, but the majority of statin drugs require activation. The other statins, lovastatin (Tocris Bioscience), simvastatin (Tocris Bioscience), atorvastatin (Crescent Chemical), were activated in ethanol followed by adjustment of pH to levels suitable for use in vivo (pH 7-8), per manufacturer guidelines. The activated stock solution was diluted with ethanol to 20× the injected dose and stored at −20° C. for up to one month. The day of injection, a 1× working solution was prepared by diluting one part of the stock solution in 18 parts sterile saline and 1 part DMSO, such that the desired dose for a 20 g mouse was given in 100 ul. Mice were injected subcutaneously twice-weekly, beginning at five weeks of age with a dose shown in Table 1. Mice were assessed per the diagram in FIG. 6.

TABLE 1
Exemplary properties of Statins used in Example 4

| | Atorvastatin | | Fluvastatin | | Lovastatin | | Simvastatin | |
|---|---|---|---|---|---|---|---|---|
| Metabolized | Cyp3A4 | | Cyp2C9 | | Cyp3A4 | | Cyp3A4 | |
| by: | | | | | | | | |
| Clearance | 1 U/0.25 hr | | 1 U/hr | | 1 U/0.2 hr | | 1 U/0.4 hr | |
| rate from | | | | | | | | |
| circulation: | | | | | | | | |
| Lipophilicity | 1.00-1.25 | | 1.00-1.25 | | 1.75-1.50 | | 1.75-1.50 | |
| (Log_{D}): | | | | | | | | |
| LD₅₀ (oral): | >5000 mg/kg | | >2000 mg/kg | | >1000 mg/kg | | >3000 mg/kg | |
| | (mouse/rat) | | (mouse) | | (mouse) | | (rat) | |
| IC₅₀ | ~6 | nM | ~8 | nM | 4.4 | nM | 13.3 | nM |
| (synthesis in | | | | | | | | |
| rat | | | | | | | | |
| micro- | | | | | | | | |
| somes): | | | | | | | | |
| Proposed | 2 | mg/kg | 3 | mg/kg | 1.5 | mg/kg | 6 | mg/kg |
| dose: | | | | | | | | |

As shown in Table 1, each of the statin drugs used in this Example has a different rate of clearance, is metabolized by different cytochrome p450 enzymes, and has a different lethal dose at which 50% of the animals die, as determined by rats (LD₅₀). In addition, each statin drug has a different half-life with lovastatin having a half-life of approximately 9 hours, atorvastatin having a half-life of approximately 14 hours, simvastatin having a half-life of approximately 2-3 hours, and fluvastatin having a half-life of approximately 96 hours.

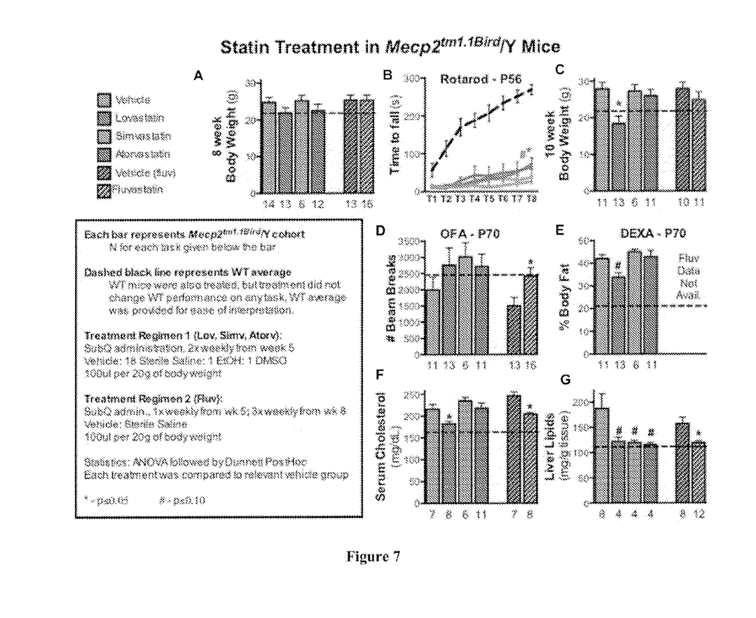

FIG. 7 shows the results of statin treatment on Mecp2 mice for 5 weeks. As shown in FIG. 7, lovastatin showed significant improvements in rotarod performance, open field activity, serum cholesterol level, and liver lipid panel as compared to vehicle control animals. In addition, lovastatin mice had significantly lower body weights after 5 weeks than any other groups. This data is particularly interesting given the short half-life of lovastatin (˜9 hours) as compared to the dosing schedule used in this Example (2× per week). Therefore, without wishing to be held to a particular theory, it is possible that lovastatin (and possibly the other statins as well) may show significantly increased effectiveness if dosed at a more frequent interval. Body weights were obtained weekly starting at 5 weeks of age prior to treatment, and ending at 10 weeks, at the end of study. Shown in FIG. 7 are the weights after 3 weeks of treatment (at 8 weeks of age, panel A) and after 5 weeks of treatment (10 weeks of age, panel C).

### EXAMPLE 5

### Abnormal Glucose Uptake in RTT Sufferers

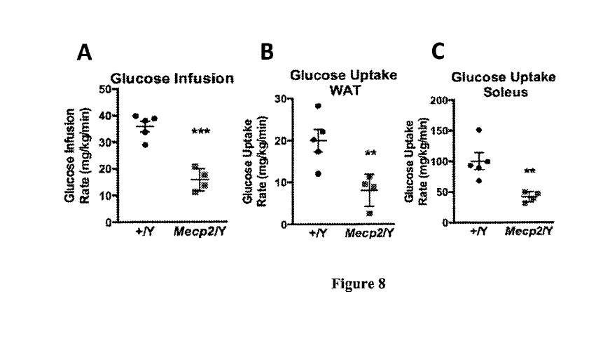

This Example, in confirmation and extension of the findings in Example 3 (see FIG. 3), shows that individuals suffering from Mecp2 dysfunction exhibit abnormal glucose uptake and insulin resistance as shown through the use of a hyperinsulemic-euglycemic clamp. Specifically, as shown in FIG. 8, Mecp2 mice are insulin resistant and suffer from metabolic syndrome.

In this Example, the implantation of the hyperinsulemic-euglycemic clamp occurred as follows. Eight week old Mecp2^{tm1.1Bird/Y} male mice were anesthetized and a midline neck incision was made to expose the jugular vein. A microcannula was inserted into the jugular vein, threaded into the right atrium, and anchored at the venotomy site. Mice were allowed to recover for 4 days with ad libitum access to water and food. Following an overnight fast, the conscious mice received a primary infusion (10 uCi) and then a constant rate intravenous infusion (0.1 uCi/min) of chromatography-purified [3-³H]-glucose using a syringe infusion pump. For determination of basal glucose production, blood samples were collected from the tail vein after 50, 55, and 60 minutes of labeled glucose infusion. After 60 minutes (to allow time for glucose to enter the blood stream and be taken up by glucose-sensitive tissues), mice received a priming bolus of insulin (40 mU/kg body weight) followed by a 2-hour insulin infusion (4 mU/Icg/min). Simultaneously, 10% glucose was infused using another infusion pump at a rate adjusted to maintain the blood glucose level at 100-140 mg/dL (euglycemia). Blood glucose concentration was measured every 10 minutes by a glucometer. At 100, 110, and 120 minutes, blood was collected to measure hepatic glucose production under clamped conditions. To estimate insulin-stimulated glucose transport activity in individual tissues 2-[¹⁴C]deoxyglucose was administered as a bolus (10 uCi) at 45 minutes before the end of the clamps. After 120 minutes, mice were euthanized and tissues were extracted. Glucose uptake in different tissues was calculated from plasma by tissue enrichment of 2-[¹⁴C]deoxyglucose by gas chromatography-mass spectrometry (GCMS).

Since each animal receives the same amount of insulin, the amount of glucose infusion required to reach a steady state of 100-140 mg/dL glucose provides an indication of whole-body insulin actions. As shown in FIG. 8, as compared to wild-type mice, Mecp2^{tm1.1Bird/Y} mice require a lower infusion of glucose to reach a steady state of 100-140 mg/dL glucose, indicating insulin resistance. Panel A shows the overall rate of glucose infusion required to reach the desired steady state levels in both wild-type and Mecp2^{tm1.1Bird/Y} mice, while panels B and C show the amount of glucose uptake in the white adipose tissue (WAT) and soleus muscle of both wild-type and Mecp2^{tm1.1Bird/Y} mice. These data confirm that Mecp2^{tm1.1Bird/Y} mice are insulin resistant and have metabolic syndrome. Without wishing to be held to a particular theory, it is possible that the overproduction of peripheral lipids in Mecp2^{tm1.1Bird/Y} mice may be at least partially responsible for the observed deficiency in glucose metabolism.

### EXAMPLE 6

### Effect of Diabetes Treatments and/or Lipid Therapies on RTT Sufferers

Given the data described in Example 5 above, showing the metabolic dysfunction in Mecp2^{tm1.1Bird/Y} mice, the effect of therapies used for treatment of metabolic disorders was assessed. Specifically, the effect of the diabetes therapeutic, metformin, the LXR agonist T0901317, and a mitochondrial uncoupler DNPME, were assessed in Mecp2^{tm1.1Bird/Y} mice. In this Example, Mecp2^{tm1.1Bird/Y} mice were subjected to the protocol described above in Example 4, including in FIG. 6 and subjected to one of vehicle, metformin, the LXR agonist T0901317, or the mitochondrial uncoupler DNPME.

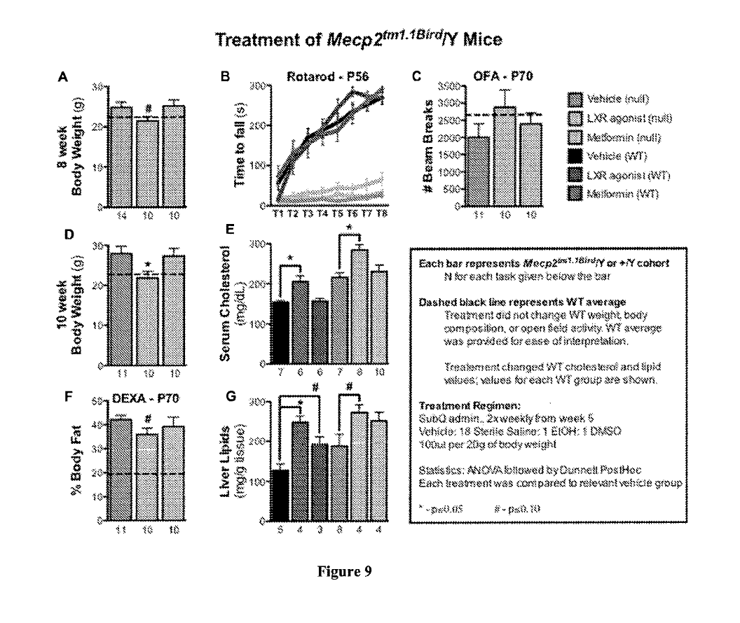

The preparation of the agents used in this Example was as follows: metformin hydrochloride, was dissolved in saline (Sigma). The day of injection, the metformin was diluted to 18 parts stock solution, 1 part DMSO, and 1 part ethanol, such that the desired dose for a 20 g mouse was given in 100 ul. Dose=30 mg/kg, injected intraperitoneally (IP). For the LXR agonist T0901317 (Caymen Chemicals) and DNPME, the drugs were dissolved in 100% DMSO and then diluted 18 parts in sterile saline, 1 part stock, 1 part ethanol. The final dose for T090317 was 25 mg/kg delivered by Sub-Q injection, and for DNPME, 5 mg/kg delivered IP. The animals were treated starting at 5 weeks of age until the age of 10 weeks, or for a period of 5 weeks. Body weights were obtained weekly starting at 5 weeks of age prior to treatment, and ending at 10 weeks, at the end of study. Shown in the FIG. 9 are the weights after 3 weeks of treatment (at 8 weeks of age, panel A) and after 5 weeks of treatment (10 weeks of age, panel D).

FIG. 9 shows the results of metformin or T090317 administration on Mecp2^{tm1.1Bird/Y} mice, as compared to vehicle administration. While metformin appeared to have little effect in this Example, T090317 improved motor performance as shown by the rotarod and open field activity assays. However, T090317 did not appear to improve peripheral fat measures. As such, without wishing to be held to a particular theory, the improvements observed in T090317 may be due to an alteration of brain lipids (e.g., cholesterol) rather than systemic lipids. In addition, it is possible that metformin may have significant effects if administered according to its FDA-recommended daily dosing schedule.

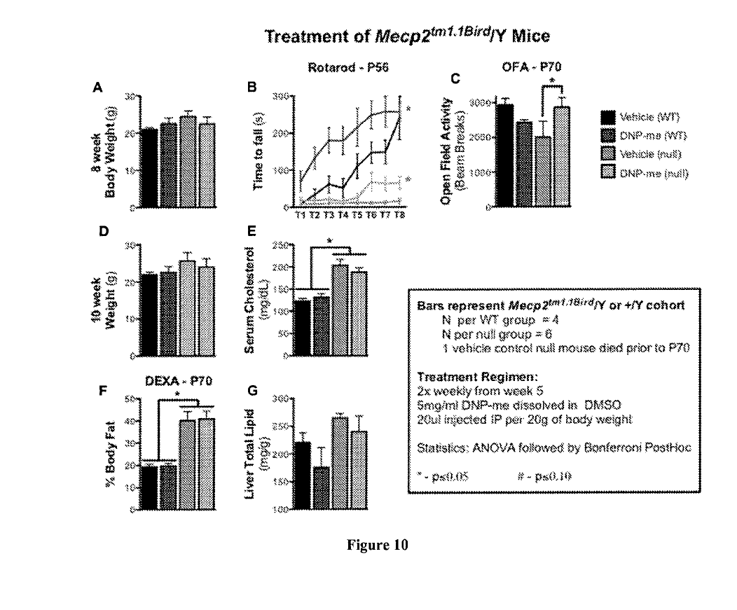

DNPME is a mitochondrial uncoupler, which uncouples energy production by ATP from glucose, and allows lipids to be used instead. Without wishing to be held to a particular theory, it is possible the administration of DNPME to sufferers of Mecp2 dysfunction causes the breakdown of lipids in the body, thus increasing the availability of certain lipids in the brain (e.g., cholesterol) or by allowing the mouse to use lipids as an energy source rather than glucose. While the mitochondrial uncoupling activity of DNPME was shown previously (see Perry R J et al., Reversal of hypertriglyceridemia, fatty liver disease, and insulin resistance by a liver-targeted mitochondrial uncoupler, 2013, Cell Metabolism, 18: 740-748), this work represents the first time that DNPME has been shown to have an effect on the state of lipid and/or cholesterol biosynthesis in the brain. As shown in FIG. 10, administration of DNPME resulted in significantly improved performance in the rotarod and OFA assays by Mecp2^{tm1.1Bird/Y} mice. It is likely that daily dosing would result in even better outcomes and that combination therapies, such as with statin drugs or other therapeutic compounds, may result in still further benefits.

This Example shows that therapeutic compounds that are shown to be efficacious in treating type II diabetes and/or lipid depositions may be attractive therapies for the treatment of Mecp2-related diseases, disorders, or conditions, such as Rett Syndrome.

In sum, the above Examples clearly demonstrate and confirm that agents or modalities that modulate lipid and/or cholesterol metabolism in the brain represent a previously unknown class of therapeutics for use in treating Mecp2-related diseases, disorders or conditions, such as Rett Syndrome. According to various embodiments, such agents are able to improve motor performance, lower lipid levels, and extend life in subjects suffering from Mecp2 dysfunction.

### Other Embodiments and Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

What is claimed is:

1. A method of treating a MECP2-associated disease, disorder, or condition comprising administering at least one agent or modality that modulates lipid and/or cholesterol metabolism in the brain to a subject in need thereof.

2. The method of claim 1, wherein the at least one agent or modality is selected from: a statin, an LXR modulator, a glucose metabolism modulator, a SREBP modulator, a PPARG modulator, and combinations thereof.

3. The method of claim 2, wherein the statin is one or more of lovastatin, simvastatin, alorvastatin, rosuvastatin, and fluvastatin.

4. The method of claim 2, wherein the LXR modulator is at least one of an oxysterol, an LXR agonist, and/or an RXR agonist.

5. The method of claim 4, wherein the LXR modulator is at least one of hypocholamide, T0901317, GW3965, SR9238, 22(R)-hydroxycholesterol, 24(S)-hydroxysterol, 27-hydroxycholesterol, cholestenoic acid and bexarotene.

6. The method of claim 2, wherein the glucose metabolism modulator is at least one of a biguanide drug and 2,4-dinitrophenol-methyl ether (DNP-ME) or derivative thereof.

7. The method of claim 6, wherein the at least one biguanide drug is selected from: metformin, proguanil, chlorproguanil.

8. The method of claim 2, wherein the SREBP modulator is at least one of fatostatin, N-(4-(2-2-propylpyridin-4-yl)thiazol-4-yl)phenyl)methanesulfonamide (FGH10019), SREBP1, and SREBP2.

9. The method of claim 2, wherein the PPARG modulator is a thiazolidinedione.

10. The method of claim 9, wherein the thiazolidinedione is at least one of rosiglitazone, pioglitazone, troglitazone, netoglitazone, rivoglitazone, and ciglitazone.

11. The method of any one of the above claims, wherein the at least one agent or modality is administered at least once per day.

12. The method of any one of the above claims, wherein the at least one agent or modality is administered at least once per week.

13. The method of any one of the above claims, wherein the at least one agent or modality is administered at least twice per week.

14. The method of any one of the above claims, wherein the at least one agent or modality is administered subcutaneously, intraperitoneally, intravenously, or orally.

15. A method of identifying and/or characterizing useful therapeutic agents for the treatment of Rett Syndrome comprising
determining the effect of a candidate therapeutic agent on one or more aspects of lipid and/or cholesterol metabolism in the brain.

16. The method of claim 15, wherein the one or more aspects of lipid and/or cholesterol metabolism is cholesterol biosynthesis.

17. The method of claim 15, wherein the one or more aspects of lipid and/or cholesterol metabolism is inhibition of 3-hydroxy-3-methyl-glutaryl-CoA reductase (HMGCR).

18. The method of claim 15, wherein the one or more aspects of lipid and/or cholesterol metabolism is inhibition of squalene monooxygenase

19. The method of any one of claims 15-18, wherein the effect of therapeutic agents on one or more aspects of lipid and/or cholesterol metabolism is assessed via one or more of a: behavioral test, cognitive test, motor function test, test of one or more physiological parameters, and combinations thereof.

20. The method of claim 19, wherein the behavioral test is selected from: acoustic startle response test, pre-pulse inhibition of startle response test, open field activity test, three chamber social interaction test, Home Cage Activity test, and/or combinations thereof.

21. The method of claim 19, wherein the motor function test is selected from: rotarod test, open field locomotor activity test, DigiGait monitoring system, and combinations thereof.

22. The method of claim 19, wherein the test of one or more physiological parameters is selected from: dual X-ray absorptiometry (DEXA) test, whole body plethysmography breathing test with methacholine challenge, glucose tolerance test, insulin tolerance test, serum cholesterol test, calorimetry test, and combination thereof.

23. A method of treating Rett Syndrome comprising administering a statin to a subject suffering from or susceptible to Rett Syndrome.

24. The method of claim 23, wherein the statin is selected from: lovastatin, simvastatin, atorvastatin, fluvastatin, and combinations thereof.

25. The method of claim 23 or 24, wherein the statin is administered at least once per day.

26. The method of claim 23 or 24, wherein the statin is administered at least once per week.

27. The method of claim 23 or 24, wherein the statin is administered at least twice per week.

28. The method of any one of claims 23-27, wherein the statin is administered subcutaneously or orally.

29. A method of treating Rett Syndrome comprising administering a glucose metabolism modulator to a subject suffering from or susceptible to Rett Syndrome.

30. The method of claim 29, wherein the glucose metabolism modulator is selected from: a biguanide drug; 2,4-dinitrophenol-methyl ether (DNP-ME), 2,4-dinitrophenol-ethyl ether (DNP-EE), 2,4-dinitrophenol-vinyl ether (DNP-VE), or a derivatives thereof; and combinations thereof.

31. The method of claim 30, wherein the biguanide drug is selected from: metformin, proguanil, chlorproguanil.

32. The method of any one of claims 29-31, wherein the glucose metabolism modulator is administered at least once per week.

33. The method of any one of claims 29-31, wherein the glucose metabolism modulator is administered at least twice per week.

34. The method of any one of claims 29-33, wherein the glucose metabolism modulator is administered subcutaneously, intraperitoneally, intravenously, or orally.
